SEQUENCE LISTING (54 sequences)

SEQ ID NO: 1 - DNA, 1863 bp, Artificial Sequence
  Description of Artificial Sequence Synthetic polynucleotide
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctcagcgaggagatgattgctgagttcaaagctgcctttgacatgtttgatgcggacggtggtggggacatcagcaccaaggagttgggcacggtgatgaggatgctgggccagaaccccaccaaagaggagctggatgccatcatcgaggaggtggacgaggatggcagcggcaccatcgacttcgaggagttcctggtgatgatggtgcgccagatgaaagaggacgccaagggcaagtctgaggaggagctggccaactgcttccgcatcttcgacaagaacgctgatgggttcatcgacatcgaggagctgggtgagattctcagggccactggggagcacgtcatcgaggaggacatagaagacctcatgaaggattcagacaagaacaatgacggccgcattgacttcgatgagttcctgaagatgatggagggtgtgcaggagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 2 - protein, 620 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLSEEMIAEFKAAFDMFDADGGGDISTKELGTVMRMLGQNPTKEELDAIIEEVDEDGSGTIDFEEFLVMMVRQMKEDAKGKSEEELANCFRIFDKNADGFIDIEELGEILRATGEHVIEEDIEDLMKDSDKNNDGRIDFDEFLKMMEGVQELMVSKGEELFTGVVPILVELDGDVNGHKFSVSGEGEGDATYGKLTLKFICTTGKLPVPWPTLVTTFGYGLMCFARYPDHMRQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSYQSALSKDPNEKRDHMVLLEFVTAAGITLGMDELYK

SEQ ID NO: 3 - DNA, 1902 bp, Artificial Sequence
  Description of Artificial Sequence Synthetic polynucleotide
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctaatggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggaactcatgaaggacggagacaagaacaacgacggccgcatcgactatgatgagttcctggagttcatgaagggtgtggaggagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 4 - protein, 633 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLMDDIYKAAVEQLTEEQKNEFKAAFDIFVLGAEDGCISTKELGKVMRMLGQNPTPEELQEMIDEVDEDGSGTVDFDEFLVMMVRCMKDDSKGKSEEELSDLFRMFDKNADGYIDLDELKIMLQATGETITEDDIEELMKDGDKNNDGRIDYDEFLEFMKGVEELMVSKGEELFTGVVPILVELDGDVNGHKFSVSGEGEGDATYGKLTLKFICTTGKLPVPWPTLVTTFGYGLMCFARYPDHMRQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSYQSALSKDPNEKRDHMVLLEFVTAAGITLGMDELYK

SEQ ID NO: 5 - DNA, 1863 bp, Artificial Sequence
  Description of Artificial Sequence Synthetic polynucleotide
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctcagcgaggagatgattgctgagttcaaagctgcctttgacatgtttgatgcggacggtggtggggacatcagcaccaaggagttgggcacggtgatgaggatgctgggccagaaccccaccaaagaggagctggatgccatcatcgaggaggtggacgaggatggcagcggcaccatcgacttcgaggagttcctggtgatgatggtgcgccagatgaaagaggacgccaagggcaagtctgaggaggagctggccaactgcttccgcatcttcgccaagaacgctgatgggttcatcgacatcgaggagctgggtgagattctcagggccactggggagcacgtcatcgaggaggacatagaagacctcatgaaggattcagacaagaacaatgacggccgcattgacttcgatgagttcctgaagatgatggagggtgtgcaggagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 6 - protein, 620 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLSEEMIAEFKAAFDMFDADGGGDISTKELGTVMRMLGQNPTKEELDAIIEEVDEDGSGTIDFEEFLVMMVRQMKEDAKGKSEEELANCFRIFAKNADGFIDIEELGEILRATGEHVIEEDIEDLMKDSDKNNDGRIDFDEFLKMMEGVQELMVSKGEELFTGVVPILVELDGDVNGHKFSVSGEGEGDATYGKLTLKFICTTGKLPVPWPTLVTTFGYGLMCFARYPDHMRQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSYQSALSKDPNEKRDHMVLLEFVTAAGITLGMDELYK

SEQ ID NO: 7 - DNA, 1908 bp, Artificial Sequence
  Description of Artificial Sequence Synthetic polynucleotide
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctaatggcgtcaatgacggaccagcaggcggaggcccgcgccttcctcagcgaggagatgattgctgagttcaaagctgcctttgacatgtttgatgcggacggtggtggggacatcagcaccaaggagttgggcacggtgatgaggatgctgggccagaaccccaccaaagaggagctggatgccatcatcgaggaggtggacgaggatggcagcggcaccatcgacttcgaggagttcctggtgatgatggtgcgccagatgaaagaggacgccaagggcaagtctgaggaggagctggccaactgcttccgcatcttcgacaagaacgctgatgggttcatcgacatcgaggagctgggtgagattctcagggccactggggagcacgtcatcgaggaggacatagaagacctcatgaaggattcagacaagaacaatgacggccgcattgacttcgatgagttcctgaagatgatggagggtgtgcaggagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 8 - protein, 635 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLMASMTDQQAEARAFLSEEMIAEFKAAFDMFDADGGGDISTKELGTVMRMLGQNPTKEELDAIIEEVDEDGSGTIDFEEFLVMMVRQMKEDAKGKSEEELANCFRIFDKNADGFIDIEELGEILRATGEHVIEEDIEDLMKDSDKNNDGRIDFDEFLKMMEGVQELMVSKGEELFTGVVPILVELDGDVNGHKFSVSGEGEGDATYGKLTLKFICTTGKLPVPWPTLVTTFGYGLMCFARYPDHMRQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSYQSALSKDPNEKRDHMVLLEFVTAAGITLGMDELYK

SEQ ID NO: 9 - DNA, 1542 bp, Artificial Sequence
  Description of Artificial Sequence Synthetic polynucleotide
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctaggccagaaccccaccaaagaggagctggatgccatcatcgaggaggtggacgaggatggcagcggcaccatcgacttcgaggagttcctggtgatgatggtgcgccagatgaaagaggacgccgagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 10 - protein, 513 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLGQNPTKEELDAIIEEVDEDGSGTIDFEEFLVMMVRQMKEDAELMVSKGEELFTGVVPILVELDGDVNGHKFSVSGEGEGDATYGKLTLKFICTTGKLPVPWPTLVTTFGYGLMCFARYPDHMRQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSYQSALSKDPNEKRDHMVLLEFVTAAGITLGMDELYK

SEQ ID NO: 11 - DNA, 2469 bp, Artificial Sequence
  Description of Artificial Sequence Synthetic polynucleotide
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctaatggcgtcaatgacggaccagcaggcggaggcccgcgccttcctcagcgaggagatgattgctgagttcaaagctgcctttgacatgtttgatgcggacggtggtggggacatcagcaccaaggagttgggcacggtgatgaggatgctgggccagaaccccaccaaagaggagctggatgccatcatcgaggaggtggacgaggatggcagcggcaccatcgacttcgaggagttcctggtgatgatggtgcgccagatgaaagaggacgccaagggcaagtctgaggaggagctggccaactgcttccgcatcttcgacaagaacgctgatgggttcatcgacatcgaggagctgggtgagattctcagggccactggggagcacgtcatcgaggaggacatagaagacctcatgaaggattcagacaagaacaatgacggccgcattgacttcgatgagttcctgaagatgatggagggtgtgcaggagctcggcggcatgtctgatgaagagaaaaagcgtcgtgcagccaccgcccgtcgtcagcacctgaagagtgctatgctccagcttgctgtcactgaaatagaaaaagaagcagctgctaaagaagtggaaaagcaaaactacctggcagagcatagccctcctctgtccctcccagggtccatgcaggaacttcaggaactgagcaaaaaacttcatgccaagatagactcagtggatgaggaaaggtatgacacagaggtgaagctacagaagactaacaaggagctggaggacctgagccagaagctgtttgacctgaggggcaagttcaagaggccacctctgcgccgggtgcgcatgtctgctgatgccatgctgcgtgccctgctgggctccaagcacaaggtcaacatggacctccgggccaacctgaagcaagtcaagaaggaggacacggagaaggagaaggacctccgcgatgtgggtgactggaggaagaacattgaggagaaatctggcatggagggcaggaagaagatgtttgaggccggcgagtccgagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 12 - protein, 822 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLMASMTDQQAEARAFLSEEMIAEFKAAFDMFDADGGGDISTKELGTVMRMLGQNPTKEELDAIIEEVDEDGSGTIDFEEFLVMMVRQMKEDAKGKSEEELANCFRIFDKNADGFIDIEELGEILRATGEHVIEEDIEDLMKDSDKNNDGRIDFDEFLKMMEGVQELGGMSDEEKKRRAATARRQHLKSAMLQLAVTEIEKEAAAKEVEKQNYLAEHSPPLSLPGSMQELQELSKKLHAKIDSVDEERYDTEVKLQKTNKELEDLSQKLFDLRGKFKRPPLRRVRMSADAMLRALLGSKHKVNMDLRANLKQVKKEDTEKEKDLRDVGDWRKNIEEKSGMEGRKKMFEAGESELMVSKGEELFTGVVPILVELDGDVNGHKFSVSGEGEGDATYGKLTLKFICTTGKLPVPWPTLVTTFGYGLMCFARYPDHMRQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSYQSALSKDPNEKRDHMVLLEFVTAAGITLGMDELYK

SEQ ID NO: 13 - DNA, 1959 bp, Artificial Sequence
  Description of Artificial Sequence Synthetic polynucleotide
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctcgctgatgccatgctgcgtgccctgctgggctccaagcacaaggtcaacggcggcgcgtcaatgacggaccagcaggcggaggcccgcgccttcctcagcgaggagatgattgctgagttcaaagctgcctttgacatgtttgatgcggacggtggtggggacatcagcaccaaggagttgggcacggtgatgaggatgctgggccagaaccccaccaaagaggagctggatgccatcatcgaggaggtggacgaggatggcagcggcaccatcgacttcgaggagttcctggtgatgatggtgcgccagatgaaagaggacgccaagggcaagtctgaggaggagctggccaactgcttccgcatcttcgacaagaacgctgatgggttcatcgacatcgaggagctgggtgagattctcagggccactggggagcacgtcatcgaggaggacatagaagacctcatgaaggattcagacaagaacaatgacggccgcattgacttcgatgagttcctgaagatgatggagggtgtgcaggagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 14 - protein, 652 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLADAMLRALLGSKHKVNGGASMTDQQAEARAFLSEEMIAEFKAAFDMFDADGGGDISTKELGTVMRMLGQNPTKEELDAIIEEVDEDGSGTIDFEEFLVMMVRQMKEDAKGKSEEELANCFRIFDKNADGFIDIEELGEILRATGEHVIEEDIEDLMKDSDKNNDGRIDFDEFLKMMEGVQELMVSKGEELFTGVVPILVELDGDVNGHKFSVSGEGEGDATYGKLTLKFICTTGKLPVPWPTLVTTFGYGLMCFARYPDHMRQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSYQSALSKDPNEKRDHMVLLEFVTAAGITLGMDELYK

SEQ ID NO: 15 - DNA, 1827 bp, Artificial Sequence
  Description of Artificial Sequence Synthetic polynucleotide
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctagacctgagccagaagctgtttgacctgaggggcaagttcaagaggccacctctgcgccgggtgcgcatgtctgctgatgccatgctgcgtgccctgctgggctccaagcacaaggtcggcagcggcagcatgctaatggcgtcaatgacggaccagcaggcggaggcccgcgccttcctcagcgaggagatgattgctgagttcaaagctgcctttgacatgtttgatgcggacggtggtggggacatcagcaccaaggagttgggcacggtgatgaggatgctgggccagaaccccaccaaagaggagctggatgccatcatcgaggaggtggacgaggatggcagcggcaccatcgacttcgaggagttcctggtgatgatggtgcgccagatgaaagaggacgccgagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 16 - protein, 608 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLDLSQKLFDLRGKFKRPPLRRVRMSADAMLRALLGSKHKVGSGSMLMASMTDQQAEARAFLSEEMIAEFKAAFDMFDADGGGDISTKELGTVMRMLGQNPTKEELDAIIEEVDEDGSGTIDFEEFLVMMVRQMKEDAELMVSKGEELFTGVVPILVELDGDVNGHKFSVSGEGEGDATYGKLTLKFICTTGKLPVPWPTLVTTFGYGLMCFARYPDHMRQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSYQSALSKDPNEKRDHMVLLEFVTAAGITLGMDELYK

SEQ ID NO: 17 - DNA, 1869 bp, Artificial Sequence
  Description of Artificial Sequence Synthetic polynucleotide
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctgctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggaactcatgaaggacggagacaagaacaacgacggccgcatcgactatgatgagttcctggagttcatgaagggtgtggaggagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 18 - protein, 622 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLLTEEQKNEFKAAFDIFVLGAEDGCISTKELGKVMRMLGQNPTPEELQEMIDEVDEDGSGTVDFDEFLVMMVRCMKDDSKGKSEEELSDLFRMFDKNADGYIDLDELKIMLQATGETITEDDIEELMKDGDKNNDGRIDYDEFLEFMKGVEELMVSKGEELFTGVVPILVELDGDVNGHKFSVSGEGEGDATYGKLTLKFICTTGKLPVPWPTLVTTFGYGLMCFARYPDHMRQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSYQSALSKDPNEKRDHMVLLEFVTAAGITLGMDELYK

SEQ ID NO: 19 - DNA, 486 bp, Homo sapiens
atggatgacatctacaaggctgcggtagagcagctgacagaagagcagaaaaatgagttcaaggcagccttcgacatcttcgtgctgggcgctgaggatggctgcatcagcaccaaggagctgggcaaggtgatgaggatgctgggccagaaccccacccctgaggagctgcaggagatgatcgatgaggtggacgaggacggcagcggcacggtggactttgatgagttcctggtcatgatggttcggtgcatgaaggacgacagcaaagggaaatctgaggaggagctgtctgacctcttccgcatgtttgacaaaaatgctgatggctacatcgacctggatgagctgaagataatgctgcaggctacaggcgagaccatcacggaggacgacatcgaggagctcatgaaggacggagacaagaacaacgacggccgcatcgactatgatgagttcctggagttcatgaagggtgtggagtag

SEQ ID NO: 20 - protein, 161 aa, Homo sapiens
MDDIYKAAVEQLTEEQKNEFKAAFDIFVLGAEDGCISTKELGKVMRMLGQNPTPEELQEMIDEVDEDGSGTVDFDEFLVMMVRCMKDDSKGKSEEELSDLFRMFDKNADGYIDLDELKIMLQATGETITEDDIEELMKDGDKNNDGRIDYDEFLEFMKGVE

SEQ ID NO: 21 - DNA, 633 bp, Homo sapiens
atggcggatgggagcagcgatgcggctagggaacctcgccctgcaccagccccaatcagacgccgctcctccaactaccgcgcttatgccacggagccgcacgccaagaaaaaatctaagatctccgcctcgagaaaattgcagctgaagactctgctgctgcagattgcaaagcaagagctggagcgagaggcggaggagcggcgcggagagaaggggcgcgctctgagcacccgctgccagccgctggagttgaccgggctgggcttcgcggagctgcaggacttgtgccgacagctccacgcccgtgtggacaaggtggatgaagagagatacgacatagaggcaaaagtcaccaagaacatcacggagattgcagatctgactcagaagatctttgaccttcgaggcaagtttaagcggcccaccctgcggagagtgaggatctctgcagatgccatgatgcaggcgctgctgggggcccgggctaaggagtccctggacctgcgggcccacctcaagcaggtgaagaaggaggacaccgagaaggaaaaccgggaggtgggagactggcggaagaacatcgatgcactgagtggaatggagggccgcaagaaaaagtttgagagctga

SEQ ID NO: 22 - protein, 210 aa, Homo sapiens
MADGSSDAAREPRPAPAPIRRRSSNYRAYATEPHAKKKSKISASRKLQLKTLLLQIAKQELEREAEERRGEKGRALSTRCQPLELTGLGFAELQDLCRQLHARVDKVDEERYDIEAKVTKNITEIADLTQKIFDLRGKFKRPTLRRVRISADAMMQALLGARAKESLDLRAHLKQVKKEDTEKENREVGDWRKNIDALSGMEGRKKKFES

SEQ ID NO: 23 - DNA, 483 bp, Homo sapiens
atgacggaccagcaggctgaggccaggtcctacctcagcgaagagatgatcgctgagttcaaggctgcctttgacatgtttgatgctgatggtggtggggacatcagcgtcaaggagttgggcacggtgatgaggatgctgggccagacacccaccaaggaggagctggacgccatcatcgaggaggtggatgaggacggcagcggcaccatcgacttcgaggagttcttggtcatgatggtgcgccagatgaaagaggacgcgaaagggaagagcgaggaggagctggccgagtgcttccgcatcttcgacaggaatgcagacggctacatcgacccggaggagctggctgagattttcagggcctccggggagcacgtgactgacgaggagatcgaatctctgatgaaagacggcgacaagaacaacgacggccgcattgacttcgacgagttcctgaagatgatggagggcgtgcagtaa

SEQ ID NO: 24 - protein, 160 aa, Homo sapiens
MTDQQAEARSYLSEEMIAEFKAAFDMFDADGGGDISVKELGTVMRMLGQTPTKEELDAIIEEVDEDGSGTIDFEEFLVMMVRQMKEDAKGKSEEELAECFRIFDRNADGYIDPEELAEIFRASGEHVTDEEIESLMKDGDKNNDGRIDFDEFLKMMEGVQ

SEQ ID NO: 25 - DNA, 492 bp, Gallus gallus
atggcgtcaatgacggaccagcaggcggaggcccgcgccttcctcagcgaggagatgattgctgagttcaaagctgcctttgacatgtttgatgcggacggtggtggggacatcagcaccaaggagttgggcacggtgatgaggatgctgggccagaaccccaccaaagaggagctggatgccatcatcgaggaggtggacgaggatggcagcggcaccatcgacttcgaggagttcctggtgatgatggtgcgccagatgaaagaggacgccaagggcaagtctgaggaggagctggccaactgcttccgcatcttcgacaagaacgctgatgggttcatcgacatcgaggagctgggtgagattctcagggccactggggagcacgtcatcgaggaggacatagaagacctcatgaaggattcagacaagaacaatgacggccgcattgacttcgatgagttcctgaagatgatggagggtgtgcagtaa

SEQ ID NO: 26 - protein, 163 aa, Gallus gallus
MASMTDQQAEARAFLSEEMIAEFKAAFDMFDADGGGDISTKELGTVMRMLGQNPTKEELDAIIEEVDEDGSGTIDFEEFLVMMVRQMKEDAKGKSEEELANCFRIFDKNADGFIDIEELGEILRATGEHVIEEDIEDLMKDSDKNNDGRIDFDEFLKMMEGVQ

SEQ ID NO: 27 - DNA, 552 bp, Gallus gallus
atgtctgatgaagagaaaaagaggagggcagccaccgcccggcgccagcacctgaagagtgctatgctccagcttgctgtcactgaaatagaaaaagaagcagctgctaaagaagtggaaaagcaaaactacctggcagagcattgccctcctctgtccctcccaggatccatgcaggaacttcaggaactgtgcaaaaagcttcatgccaagatagactcagtggatgaggaaaggtatgacacagaggtgaagctacagaagactaacaaggagctggaggacctgagccagaagctgtttgacctgaggggcaagttcaagaggccacctctgcgccgggtgcgcatgtctgctgatgccatgctgcgtgccctgctgggctccaagcacaaggtcaacatggacctccgggccaacctgaagcaagtcaagaaggaggacacggagaaggagaaggacctccgcgatgtgggtgactggaggaagaacattgaggagaaatctggcatggagggcaggaagaagatgtttgaggccggcgagtcctaa

SEQ ID NO: 28 - protein, 183 aa, Gallus gallus
MSDEEKKRRAATARRQHLKSAMLQLAVTEIEKEAAAKEVEKQNYLAEHCPPLSLPGSMQELQELCKKLHAKIDSVDEERYDTEVKLQKTNKELEDLSQKLFDLRGKFKRPPLRRVRMSADAMLRALLGSKHKVNMDLRANLKQVKKEDTEKEKDLRDVGDWRKNIEEKSGMEGRKKMFEAGES

SEQ ID NO: 29 - DNA, 486 bp, Gallus gallus
atggatgacatctataaggcggcggttgagcagttgacagaagaacaaaaaaatgagtttaaggctgccttcgacatcttcgtgctgggggcagaggatggctgcatcagcaccaaggagctggggaaggtgatgaggatgctggggcagaaccccacccctgaggagctgcaggagatgattgatgaggtggatgaggatggcagtggcactgtggactttgatgagttccttgttatgatggttcggtgtatgaaagatgacagcaaaggaaaaactgaagaggagctctcagatctcttcaggatgtttgataagaatgctgatggctacatcgatcttgaggaactgaagatcatgctacaggcaactggagagacgatcactgaggatgacatagaagaactgatgaaagatggggacaaaaacaatgatggcaggattgactatgacgagttcctggagttcatgaagggagttgaataa

SEQ ID NO: 30 - protein, 161 aa, Gallus gallus
MDDIYKAAVEQLTEEQKNEFKAAFDIFVLGAEDGCISTKELGKVMRMLGQNPTPEELQEMIDEVDEDGSGTVDFDEFLVMMVRCMKDDSKGKTEEELSDLFRMFDKNADGYIDLEELKIMLQATGETITEDDIEELMKDGDKNNDGRIDYDEFLEFMKGVE

SEQ ID NO: 31 - DNA, 1878 bp, Artificial Sequence
  Description of Artificial Sequence Synthetic polynucleotide
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctgagcgatgaattgactaaggagcaaactgcattactacgtaatgcatttaatgcttttgaccctgaaaaaaatggatatatcaacacagctatggtgggtacgatacttagcatgttgggtcatcaacttgatgatgcaactcttgctgacattatcgctgaagtcgatgaggatggttcgggccaaatcgaatttgaagaatttaccaccctggcagcccgcttccttgtggaagaggacgctgaagctatgatggctgaattgaaggaagctttccgcctttacgacaaagaaggaaatggatatataactactggtgttcttcgtgaaatcctgcgcgaactagacgataaattgacaaatgacgacctggacatgatgattgaggaaattgattccgatggatcgggtactgttgattttgatgaatttatggaagtaatgaccggtggcgacgacgagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 32 - protein, 625 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLSDELTKEQTALLRNAFNAFDPEKNGYINTAMVGTILSMLGHQLDDATLADIIAEVDEDGSGQIEFEEFTTLAARFLVEEDAEAMMAELKEAFRLYDKEGNGYITTGVLREILRELDDKLTNDDLDMMIEEIDSDGSGTVDFDEFMEVMTGGDDELMVSKGEELFTGVVPILVELDGDVNGHKFSVSGEGEGDATYGKLTLKFICTTGKLPVPWPTLVTTFGYGLMCFARYPDHMRQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSYQSALSKDPNEKRDHMVLLEFVTAAGITLGMDELYK

SEQ ID NO: 33 - DNA, 1866 bp, Artificial Sequence
  Description of Artificial Sequence Synthetic polynucleotide
atggtgagcaagggcgaggagctgttcaccggggtggtgcccatcctggtcgagctggacggcgacgtaaacggccacaggttcagcgtgtccggcgagggcgagggcgatgccacctacggcaagctgaccctgaagttcatctgcaccaccggcaagctgcccgtgccctggcccaccctcgtgaccaccctgacctggggcgtgcagtgcttcagccgctaccccgaccacatgaagcagcacgacttcttcaagtccgccatgcccgaaggctacgtccaggagcgtaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacatcagccacaacgtctatatcaccgccgacaagcagaagaacggcatcaaggcccacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgcccgcatgctgactaaggagcaaactgcattactacgtaatgcatttaatgcttttgaccctgaaaaaaatggatatatcaacacagctatggtgggtacgatacttagcatgttgggtcatcaacttgatgatgcaactcttgctgacattatcgctgaagtcgatgaggatggttcgggccaaatcgaatttgaagaatttaccaccctggcagcccgcttccttgtggaagaggacgctgaagctatgatggctgaattgaaggaagctttccgcctttacgacaaagaaggaaatggatatataactactggtgttcttcgtgaaatcctgcgcgaactagacgataaattgacaaatgacgacctggacatgatgattgaggaaattgattccgatggatcgggtactgttgattttgatgaatttatggaagtaatgaccggtggcgacgacgagctcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcggctacggcctgatgtgcttcgcccgctaccccgaccacatgcgccagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggccaacttcaagatccgccacaacatcgaggacggcagcgtgcagctcgccgaccactaccagcagaacacccccatcggcgacggccccgtgctgctgcccgacaaccactacctgagctaccagtccgccctgagcaaagaccccaacgagaagcgcgatcacatggtcctgctggagttcgtgaccgccgccgggatcactctcggcatggacgagctgtacaagtaa

SEQ ID NO: 34 - protein, 621 aa, Artificial Sequence
  Description of Artificial Sequence Synthetic polypeptide
MVSKGEELFTGVVPILVELDGDVNGHRFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKAHFKIRHNIEDGSVQLADHYQQNTPIGDGPVLLPDNHYLSTQSALSKDPNEKRDHMVLLEFVTAARMLTKEQTALLRNAFNAFDPEKNGYINTAMVGTILSMLGHQLDDATLADIIAEVDEDGSGQIEFEEFTTLAARFLVEEDAEAMMAELKEAFRLYDKEGNGYITTGVLREILRELDDKLTNDDLDMMIEEIDSDGSGTVDFDEFMEVMTGGDDELMVSKGEELFTGVVPILVELDGDVNGHKFSVSGEGEGDATYGKLTLKFICTTGKLPVPWPTLVTTFGYGLMCFARYPDHMRQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSYQSALSKDPNEKRDHMVLLEFVTAAGITLGMDELYK

SEQ ID NO: 35 - DNA, 465 bp, Drosphila melanogaster
atgagcgatgaattgactaaggagcaaactgcattactacgtaatgcatttaatgcttttgaccctgaaaaaaatggatatatcaacacagctatggtgggtacgatacttagcatgttgggtcatcaacttgatgatgcaactcttgctgacattatcgctgaagtcgatgaggatggttcgggccaaatcgaatttgaagaatttaccaccctggcagcccgcttccttgtggaagaggacgctgaagctatgatggctgaattgaaggaagctttccgcctttacgacaaagaaggaaatggatatataactactggtgttcttcgtgaaatcctgcgcgaactagacgataaattgacaaatgacgacctggacatgatgattgaggaaattgattccgatggatcgggtactgttgattttgatgaatttatggaagtaatgaccggtggcgacgactaa

SEQ ID NO: 36 - protein, 154 aa, Drosphila melanogaster
MSDELTKEQTALLRNAFNAFDPEKNGYINTAMVGTILSMLGHQLDDATLADIIAEVDEDGSGQIEFEEFTTLAARFLVEEDAEAMMAELKEAFRLYDKEGNGYITTGVLREILRELDDKLTNDDLDMMIEEIDSDGSGTVDFDEFMEVMTGGDD

SEQ ID NO: 37 - DNA, 468 bp, Drosphila melanogaster
atggacaacattgacgaagacctgacccccgagcagattgccgttctgcagaaggcattcaacagcttcgaccaccagaagaccggcagtatccccaccgaaatggtggccgatatcctccgtcttatgggtcagcccttcgacaggcagatccttgacgagctgatgcacgaggtcgatgaggacaaatccggtcgcctggagttcgaggagttcgtccagctggctgccaagttcatcgtagaggaggatgatgaggccatgcagaaggacgtgcgcgaggctttccgtctgtacgacaagcagggcaatggctacattcccacctcctgcctgaaggagatcctcaaggaactggacgaccagctgaccgaacaggagctcgacatcatgattgaggaaatcgattccgacggctctggcaccgttgattttgatgaattcatggagatgatgactggcgagtaa

SEQ ID NO: 38 - protein, 155 aa, Drosphila melanogaster
MDNIDEDLTPEQIAVLQKAFNSFDHQKTGSIPTEMVADILRLMGQPFDRQILDELMHEVDEDKSGRLEFEEFVQLAAKFIVEEDDEAMQKDVREAFRLYDKQGNGYIPTSCLKEILKELDDQLTEQELDIMIEEIDSDGSGTVDFDEFMEMMTGE

SEQ ID NO: 39 - DNA, 468 bp, Drosphila melanogaster
atgagcagcgtcgatgaagatcttacacccgagcagatcgccgtgctccagaaggcgttcaacagcttcgatcaccagaagactggctccatccccaccgagatggtcgccgacatcctgcgcctgatgggtcagcccttcgacaagaagatcctggaggaactgatcgaggaggtcgatgaggacaagtccggtcgcttggaattcggcgagttcgtccagctggctgccaagttcatcgtggaggaggatgcggaggccatgcagaaggagctggccgaggcgttccgtttgtacgataagcagggcaatggcttcattcccaccacctgcctgaaggagatcctcaaggagctggacgaccagctgaccgaacaggagctggacattatgatcgaggagatcgattccgatggctccggtacagtggatttcgatgaattcatggagatgatgactggcgagtaa

SEQ ID NO: 40 - protein, 155 aa, Drosphila melanogaster
MSSVDEDLTPEQIAVLQKAFNSFDHQKTGSIPTEMVADILRLMGQPFDKKILEELIEEVDEDKSGRLEFGEFVQLAAKFIVEEDAEAMQKELAEAFRLYDKQGNGFIPTTCLKEILKELDDQLTEQELDIMIEEIDSDGSGTVDFDEFMEMMTGE

SEQ ID NO: 41 - DNA, 1833 bp, Artificial Sequence
  Description of Artificial Sequence Synthetic polynucleotide
atgggccccgccatgaagatcgagtgccgcatcaccggcaccctgaacggcgtggagttcgagctggtgggcggcggagagggcacccccgagcagggccgcatgaccaacaagatgaagagcaccaagggcgccctgaccttcagcccctacctgctgagccacgtgatgggctacggcttctaccacttcggcacctaccccagcggctacgagaaccccttcctgcacgccatcaacaacggcggctacaccaacacccgcatcgagaagtacgaggacggcggcgtgctgcacgtgagcttcagctaccgctacgaggccggccgcgtgatcggcgacttcaaggtggtgggcaccggcttccccgaggacagcgtgatcttcaccgacaagatcatccgcagcaacgccaccgtggagcacctgcaccccatgggcgataacgtgctggtgggcagcttcgcccgcaccttcagcctgcgcgacggcggctactacagcttcgtggtggacagccacatgcacttcaagagcgccatccaccccagcatcctgcagaacgggggccccatgttcgccttccgccgcgtggaggagctgcacagcaacaccgagctgggcatcgtggagtaccagcacgccttcaagaccccgatcgcattcgcccgcatgctcagcgaggagatgattgctgagttcaaagctgcctttgacatgtttgatgcggacggtggtggggacatcagcaccaaggagttgggcacggtgatgaggatgctgggccagaaccccaccaaagaggagctggatgccatcatcgaggaggtggacgaggatggcagcggcaccatcgacttcgaggagttcctggtgatgatggtgcgccagatgaaagaggacgccaagggcaagtctgaggaggagctggccaactgcttccgcatcttcgacaagaacgctgatgggttcatcgacatcgaggagctgggtgagattctcagggccactggggagcacgtcatcgaggaggacatagaagacctcatgaaggattcagacaagaacaatgacggccgcattgacttcgatgagttcctgaagatgatggagggtgtgcaggagctcatgtccagcggcgccctgctgttccacggcaagatcccctacgtggtggagatggagggcaatgtggatggccacaccttcagcatccgcggcaagggctacggcgatgccagcgtgggcaaggtggatgcccagttcatctgcaccaccggcgatgtgcccgtgccctggagcaccctggtgaccaccctgacctacggcgcccagtgcttcgccaagtacggccccgagctgaaggatttctacaagagctgcatgcccgatggctacgtgcaggagcgcaccatcaccttcgagggcgatggcaatttcaagacccgcgccgaggtgaccttcgagaatggcagcgtgtacaatcgcgtgaagctgaatggccagggcttcaagaaggatggccacgtgctgggcaagaatctggagttcaatttcaccccccactgcctgtacatctggggcgatcaggccaatcacggcctgaagagcgccttcaagatctgccacgagatcaccggcagcaagggcgatttcatcgtggccgatcacacccagatgaatacccccatcggcggcggccccgtgcacgtgcccgagtaccaccacatgagctaccacgtgaagctgagcaaggatgtgaccgatcaccgcgataatatgagcctgaaggagaccgtgcgcgccgtggattgccgcaagacctacctgtga

SEQ ID NO: 42 - protein, 610 aa, Artificial Sequence
  Description of Artificial Sequence Synthetic polypeptide
MGPAMKIECRITGTLNGVEFELVGGGEGTPEQGRMTNKMKSTKGALTFSPYLLSHVMGYGFYHFGTYPSGYENPFLHAINNGGYTNTRIEKYEDGGVLHVSFSYRYEAGRVIGDFKVVGTGFPEDSVIFTDKIIRSNATVEHLHPMGDNVLVGSFARTFSLRDGGYYSFVVDSHMHFKSAIHPSILQNGGPMFAFRRVEELHSNTELGIVEYQHAFKTPIAFARMLSEEMIAEFKAAFDMFDADGGGDISTKELGTVMRMLGQNPTKEELDAIIEEVDEDGSGTIDFEEFLVMMVRQMKEDAKGKSEEELANCFRIFDKNADGFIDIEELGEILRATGEHVIEEDIEDLMKDSDKNNDGRIDFDEFLKMMEGVQELMSSGALLFHGKIPYVVEMEGNVDGHTFSIRGKGYGDASVGKVDAQFICTTGDVPVPWSTLVTTLTYGAQCFAKYGPELKDFYKSCMPDGYVQERTITFEGDGNFKTRAEVTFENGSVYNRVKLNGQGFKKDGHVLGKNLEFNFTPHCLYIWGDQANHGLKSAFKICHEITGSKGDFIVADHTQMNTPIGGGPVHVPEYHHMSYHVKLSKDVTDHRDNMSLKETVRAVDCRKTYL

SEQ ID NO: 43 - protein, 5 aa, Artificial Sequence
  Description of Artificial Sequence Synthetic peptide
GGSGG

SEQ ID NO: 44 - protein, 18 aa, Artificial Sequence
  Description of Artificial Sequence Synthetic peptide
MLLSVPLLLGLLGLAAAD

SEQ ID NO: 45 - protein, 4 aa, Artificial Sequence
  Description of Artificial Sequence Synthetic peptide
KDEL

SEQ ID NO: 46 - protein, 20 aa, Artificial Sequence
  Description of Artificial Sequence Synthetic peptide
MLCCMRRTKQVEKNDEDQKI

SEQ ID NO: 47 - protein, 20 aa, Artificial Sequence
  Description of Artificial Sequence Synthetic peptide
KLNPPDESGTGCMSCKCVLS

SEQ ID NO: 48 - protein, 12 aa, Artificial Sequence
  Description of Artificial Sequence Synthetic peptide
VYEKLSSIESDV

SEQ ID NO: 49 - protein, 19 aa, Artificial Sequence
  Description of Artificial Sequence Synthetic peptide
MQAATLPLDNISYRRESAI

SEQ ID NO: 50 - DNA, 13 bp, Artificial Sequence
  Description of Artificial Sequence Synthetic oligonucleotide
gccgccaccatgg

SEQ ID NO: 51 - protein, 20 aa, Artificial Sequence
  Description of Artificial Sequence Synthetic peptide
KLNPPDESGPGCMSCKCVLS

SEQ ID NO: 52 - protein, 20 aa, Artificial Sequence
  Description of Artificial Sequence Synthetic peptide
MGCCMRRTKQVEKNDEDQKI

SEQ ID NO: 53 - protein, 6 aa, Artificial Sequence
  Description of Artificial Sequence Synthetic peptide
GGTGGS

SEQ ID NO: 54 - DNA, 15 bp, Artificial Sequence
  Description of Artificial Sequence Synthetic oligonucleotide
gccgccaccatggcc

(19) United States
(12) Patent Application Publication
(10) Pub. No.: US 2009/0035788 A1
(43) Pub. Date: Feb. 5, 2009

(54) Genetically encoded bioindicators of calcium-ions

(75) Inventors: Oliver Griesbeck, Munich (DE); Nicola Heim, Munich (DE)

Correspondence Address: FOLEY AND LARDNER LLP;SUITE 500, 3000 K STREET NW, WASHINGTON, DC, 20007, US

(73) Assignee: Max-Planck-Gesellschaft zur Forderung der Wissenschaften e.V.

(21) Appl. No.: 10/567,091

(22) PCT Filed: Aug. 4, 2004
(86) PCT No.: PCT/EP04/08739

§ 371 Date: Mar. 7, 2008

(30) Foreign Application Priority Data
Aug. 4, 2003 (EP) 03016691.2

(51) Int. Cl. G01N 33/566; C07K 14/00; C12N 15/11; C12N 15/85; C12N 5/10; A01K 67/027

(52) U.S. Cl. 435/7.2; 530/350; 536/23.1; 435/320.1; 435/325; 800/13

(57) ABSTRACT

The present invention relates to novel types of cellular calcium probes that are based on Troponin C and two chromophors suitable for FRET (fluorescence resonance energy transfer). The Troponin C-based calcium sensors of the invention function in diverse subcellular environments, for example even when tethered to a cellular membrane. The invention further provides nucleic acid constructs encoding the calcium probes of the invention, expression constructs, host cells and transgenic animals. Furthermore, methods for the detection of changes of local calcium concentrations and for detecting the binding of a small molecule to fragments of Troponin C are provided.

### BACKGROUND OF THE INVENTION

The use of genetically encoded fluorescent indicators for visualizing cellular calcium levels promises many advantages over fluorescent Ca-indicating dyes that have to be applied externally. Genetically encoded indicators are generated in situ inside cells after transfection, do not require cofactors, can in theory be specifically targeted to cell organelles and cellular microenvironments and do not leak out of cells during longer recording sessions. Furthermore, they should be expressible within intact tissues of transgenic organisms and thus should solve the problem of loading an indicator dye into tissue, while allowing to label specific subsets of cells of interest (for review see Zhang J., et al. “Creating new fluorescent probes for cell biology.” Nat. Rev. Mol. Biol. 3, 906-918 (2002)).

Two classes of GFP-based calcium indicators have been described so far: first, ratiometric indicators termed “Cameleons” consisting of a pair of fluorescent proteins engineered for fluorescence resonance energy transfer (FRET) carrying the calcium binding protein calmodulin as well as a calmodulin target peptide sandwiched between the GFPs (see for example Miyawaki, A. et al. “Fluorescent indicators for Ca²⁺ based on green fluorescent proteins and calmodulin.” Nature 388, 882-887 (1997); Miyawaki, A. et al. “Dynamic and quantitative calcium measurements using improved cameleons.” Proc. Natl. Acad. Sci. USA 96, 2135-2140 (1999) and Truong et al. “FRET-based in vivo Ca²⁺ imaging by a new calmodulin-GFP fusion molecule.” Nat. Struct. Biol. 8, 1069-1073 (2001)). Second, various non-ratiometric indicators with calmodulin directly inserted into a single fluorescent protein (see Baird, G. S. et al. “Circular permutation and receptor insertion within green fluorescent proteins.” Proc. Natl. Acad USA 96, 11241-11246 (1999); Nagai, T. et al. “Circularly permuted green fluorescent proteins engineered to sense Ca²⁺.” Proc. Natl. Acad Sci. USA 98, 3197-3202 (2001); Nakai, J. et al. “A high signal-to-noise Ca²⁺ probe composed of a single green fluorescent protein.” Nat. Biotechnol. 19, 137-141 (2001); and Griesbeck, O. et al. “Reducing the environmental sensitivity of yellow fluorescent protein: mechanism and applications.” J. Biol. Chem. 276, 29188-29194 (2001)).

However, calmodulin-based indicators show deficiencies in certain applications, e.g. they display only a reduced dynamic range in transgenic invertebrates compared to in vitro data of the purified indicator proteins and acute transfections (see Reiff, D. F. et al. “Differential regulation of active zone density during long-term strengthening of Drosophila neuromuscular junctions.” J. Neurosci. 22, 9399-9409; Kerr R. et al. “Optical imaging of calcium transients in neurons and pharyngeal muscle of C. elegans.” Neuron 26, 583-594; and Fiala et al. “Genetically expressed cameleon in Drosophila melanogaster is used to visualize olfactory information in projection neurons.” Curr. Biol. 12, 1877-1884 (2002)). Further, they fail to show calcium responses when targeted to certain sites within cells. No useful transgenic expression in mammals has been reported yet. Calmodulin is an ubiquitous signal protein in cell metabolism and thus under stringent regulation involving a plethora of calmodulin-binding proteins (for review see Jurado, L. A. et al. “Apocalnodulin.” Physiol. Rev. 79, 661-682 (1999)). It activates numerous kinases and phosphatases, modulates ion channels (Saimi, Y. & Kung, C. “Calmodulin as an ion channel subunit.” Ann. Rev. Physiol. 64, 289-311 (2002) and is itself extensively phosphorylated by multiple protein serine/threonine kinases and protein tyrosine kinases (Benaim, G. & Villalobo, A. “Phosphorylation of calmodulin.” Eur. J. Biochem. 269, 3619-3725 (2002).

The present inventors therefore explored ways of constructing new types of calcium probes with more specialized calcium binding proteins that are minimally influenced by the cellular regulatory protein network.

### SUMMARY OF THE INVENTION

Troponin C (TnC or TNC) is a dumbbell-shaped calcium binding protein with two globular domains connected by a central linker. It was found that novel types of calcium probes that are based on Troponin C are superior for dynamic imaging within live cells than prior art genetic calcium sensors. In particular, the calcium sensors based on Troponin C function in subcellular environments in which prior art calcium sensors have demonstrated only poor behaviour, for example when tethered to a cellular membrane. Moreover, the novel Troponin-C-based calcium sensors can be used in a multitude of cell types and even in transgenic animals, which is a further advantage compared with prior art Calcium sensors. Moreover, the Troponin-C-based calcium sensors of the invention do not interfere with intracellular Ca-signalling, in particular, they do not interfere with the important calmodulin pathway. The Troponin-C-based calcium sensors do not show any sign of unfavourable aggregation and have the further advantage that they do not interact in an unfavourable way with cytosolic components.

This invention therefore relates to modified polypeptides comprising three functional components: a first chromophor of a donor-acceptor-pair for FRET, a calcium-binding polypeptide with an identity of at least 80% to a 30 amino acid long polypeptide sequence of human Troponin C or chicken skeletal muscle Troponin C or drosophila troponin C isoform 1, and a second chromophor of a donor-acceptor-pair for FRET. Such modified calcium-binding polypeptides function as superior intracellular calcium sensors because upon calcium binding the calcium-binding polypeptide changes its conformation leading to a spatial redistribution of the two chromophores of the polypeptide of the invention. This spatial redistribution can then be detected by a change of the fluorescence properties of the overall polypeptide. Another aspect of the invention relates to nucleic acid molecules comprising a nucleotide sequence encoding a fusion polypeptide, where both the first chromophore and the second chromophore of the donor-acceptor-pair for FRET of the modified calcium-binding polypeptide of the invention are themselves polypeptides. The functionality of the above mentioned modified polypeptides and fusion proteins can readily be determined by assaying the respective molecule for its Ca-binding ability as described further below. Another aspect of the invention relates to recombinant expression vectors and host cells comprising the nucleic acid molecules of the inventions. In yet another aspect the invention provides a method for the detection of changes in local calcium concentrations. In a further aspect the invention provides a method for detecting the binding of a small chemical compound or a polypeptide to a calcium-binding polypeptide with a homology of at least 80% over a stretch of 30 amino acids to human Troponin C or chicken skeletal muscle Troponin C or drosophila troponin C isoform 1. The modified polypeptides of the invention are useful for the detection of local calcium concentrations, particularly local calcium concentration changes occurring close to a cellular membrane.

### DEFINITIONS

A “polypeptide” as used herein is a molecule comprising more than 30, and in particular more than 35, 40, 45 or even more than 50 amino acids, but less than 10,000, in particular less than 9,000, 8,000, 7,000, 6,000, 5,000, 4,000, 3,000, or 2,000, most preferably less than 1,500 amino acids. Polypeptides are usually linear amino acid polymers, wherein the individual amino acids are linked to one another via peptide bonds. Also, polypeptides which contain a low percentage, e.g. less than 5%, 3% or even only up to 1% of modified or non-natural amino acids, are encompassed. Polypeptides can be further modified by chemical modification, e.g. by phosphorylation of serine, threonine, or tyrosine residues, or by glycosylation, e.g. of asparagines or serine residues.

“Peptide” as used herein is a molecule comprising less than 30 amino acids, but preferably more than 4, 5, 6, 7, 8, or even more than 9 amino acids.

A “modified polypeptide” is a polypeptide which is not encoded as such by the genome of a naturally occurring species, in particular a polypeptide that is not identical to one of those polypeptides of the gene bank database as of Jul. 28, 2003 with a naturally occurring species identified as its source. This means that a “modified” polypeptide does not occur as such in nature, but can be, and in particular was, produced by laboratory manipulations, such as genetic engineering techniques or chemical coupling of other molecules to a polypeptide. Examples of modified polypeptides are mutant polypeptides, in particular deletions, truncations, multiple substitutions, and fusion polypeptides, which at one stage were produced by genetic engineering techniques.

A polypeptide is a “calcium-binding polypeptide” if it has a Kd for Ca²⁺ of lower than 800 μM, preferably lower than 600 μM and most preferably from 50 nM to 400 μM. A method for determining the Kd will be described below.

A polypeptide has “at least X % identity with” human Troponin C, SEQ ID NO. 20 or 24, or chicken skeletal muscle Troponin C, SEQ ID NO. 26, or drosophila Troponin C, SEQ ID NO. 35, 37, or 39, if, when a 30 amino acid stretch of its polypeptide sequence is aligned with the best matching sequence of human Troponin C or chicken skeleton muscle Troponin C or drosophila troponin C isoform 1, the amino acid identity between those two aligned sequences is X %. X can be 80 or more. For example, the corresponding polypeptide sequences in Troponin C molecules from other metazoan species, preferably other chordate species and more preferably other mammalian species, provide a source for such highly homologous polypeptides, which can substitute in the modified polypeptides of the invention for the corresponding sequences of human Troponin C or chicken skeleton muscle Troponin C or drosophila troponin C isoform 1. Preferably X is 85 or more, more preferably 90 or more, or most preferably 95 or more. It is to be understood that the case of sequence identity, that is 100% identity, is included.

Preferably, the nature of the amino acid residue change by which the polypeptide with at least X % identity to one of the reference sequences differs from said reference sequence is a semiconservative and more preferably a conservative amino acid residue exchange.

| Amino acid | Conservative substitution | Semi-conservative substitution |
|---|---|---|
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q; |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V; I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y; H | L; M; I; V; C |
| Y | F; W; H | L; M; I; V; C |

Changing from A, F, H, I, L, M, P, V, W or Y to C is semiconservative if the new cysteine remains as a free thiol. Changing from M to E, R or K is semiconservative if the ionic tip of the new side group can reach the protein surface while the methylene groups make hydrophobic contacts. Changing from P to one of K, R, E or D is semiconservative, if the side group is on the surface of the protein. Furthermore, the skilled person will appreciate that Glycines at sterically demanding positions should not be substituted and that P should not be introduced into parts of the protein which have an alpha-helical or a beta sheet structure. Preferably, the above mentioned 30 amino acid stretch comprises a region with the above mentioned sequence identity with polypeptide sequences corresponding to amino acids 3 to 28, amino acids 28 to 40, 65 to 76, 105 to 116, or 141 to 152 of hTNNC1, or amino acids 24 to 35, 57 to 68, 97 to 108, and 133 to 144 of Drosophila Troponin C isoform 1, which regions contain loops with Ca-binding capabilities.

As used herein, “FRET” relates to the phenomenon known as “fluorescence resonance energy transfer”. The principle of FRET has been described for example in J. R. Lakowicz, “Principles of Fluorescence Spectroscopy”, 2^{nd} Ed. Plenum Press, New York, 1999. Briefly, FRET can occur if the emission spectrum of a first chromophore (donor chromophore or FRET-donor) overlaps with the absorption spectrum of a second chromophore (acceptor chromophore or FRET-acceptor), so that excitation by lower-wavelength light of the donor chromophore is followed by transfer of part of the excitation energy to the acceptor chromophore. A prerequisite for this phenomenon is the very close proximity of both chromophores. A result of FRET is the decrease/loss of emission by the donor chromophore while at the same time emission by the acceptor chromophore is observed. A pair of 2 chromophores which can interact in the above described manner is called a “donor-acceptor-pair” for FRET.

A “chromophore” as used herein is that part of a molecule responsible for its light-absorbing and light-emitting properties. A chromophore can be an independent chemical entity. Chromophores can be low-molecular substances, for example, the indocyanin chromophores CY3, CY3.5, Cy5, Cy7 (available from Amersham International plc, GB), fluorescein and coumarin (for example, from Molecular Probes). But chromophores can also be fluorescent proteins, like P4-3, EGFP, S65T, BFP, CFP, YFP, Cop-Green (ppluGFP2) and Phi-Yellow (the latter two available from Evrogen) to name but a few. The latter are also commercially available in a variety of forms, for example in the context of expression constructs.

“Human Troponin C” (hTnC or hTNC) comes in two forms: Troponin C from skeletal muscle, which is a 160 amino acid polypeptide with the Swissprot Accession Number P02585, and Troponin C from cardiac muscle, which is a 161 amino acid polypeptide with the Swissprot Accession Number P₀₂₅₉₀. Troponin C in chicken also comes in two forms, a form from cardiac muscle and a form from skeletal muscle. Troponin C from chicken skeletal muscle is also sometimes used herein and is a 163 amino acid polypeptide with the Swissprot Accession Number P₀₂₅₈₈ and is herein sometimes referred to as “cs-Troponin C” or “csTnC”. Troponin C from chicken cardiac muscle is as defined in SEQ ID NO: 30. Troponin C in the fruit fly Drosophila melanogaster comes in 3 isoforms; isoform 1 with Swissprot Accession Number P47947 is present only in adult fly muscles, isoform 2 (Swissprot Accession Number P47948) is found almost exclusively in larval muscles, and isoform 3 (Swissprot Accession Number P47949) is present in both larval and adult muscles. Drosophila troponin C isoform 1 (also called TPC1_DROME; SEQ ID NO. 36) is a polypeptide 154 amino acids long and originates from the gene called TpnC41C or TnC41C. As used herein, the human Troponin C from cardiac muscle is sometimes called “hTNNC1” or “hcardTnC”, while human Troponin C from skeletal muscle is sometimes called “hTNNC2” or “hsTnC”. The structure of hTNNC1 is as follows: A helical region extending from amino acid 3 to amino acid 11 is followed by a second helical region from amino acid 14 to amino acid 28. The region from amino acid 28 to amino acid 40 is an ancestral calcium site which in its present form no longer binds calcium ions. The three calcium-binding regions in hTNNC1 are the EF-hand loop 2 extending from amino acid 65 to amino acid 76, EF-hand loop 3 from amino acid 105 to amino acid 116, and EF-hand loop 4 extending from amino acid 141 to amino acid 152. The structure of drosophila troponin C isoform 1 also comprises four EF-hand domains; the second and the fourth loop regions of the EF-hands (amino acids 57 to 68 and 133 to 144) are responsible for calcium binding whereas loop regions 1 and 3 (amino acids 24 to 35 and 97 to 108, respectively) form ancestral calcium sites that have lost their calcium binding capabilities.

The three best performing indicator constructs based on troponin C variants were given the names TN-humTnC for an indicator using the human cardiac troponin C (hcardTnC, SEQ ID NO. 3 and 4) as calcium binding moiety, TN-L15 for an indicator using a truncated version (amino acids 15-163) of the chicken skeletal muscle troponin C (csTnC, SEQ ID NO. 1 and 2) as calcium binding moiety, and TN-TPC1-L5 for an indicator using a truncated version (amino acids 5-154) of Drosophila melanogaster troponin C isoform 1 (TnC41C, SEQ ID NO. 35 and 36).

EF-hands are a type of calcium-binding domain shared among many calcium-binding proteins. This type of domain consists of a twelve-residue loop flanked on both sides by a twelve residue alphahelical domain. In an EF-hand the calcium ion is coordinated in a pentagonal-bipyramidal configuration. The six residues involved in the calcium-binding are in positions 1, 3, 5, 7, 9 and 12 of the twelve-residue loop. The invariant Glu or Asp residues at position 12 provide two oxygens for liganding Ca²⁺-ions and work as a bidentate ligand in the coordination of Ca²⁺.

As used herein, a “glycine-rich linker” comprises a peptide sequence with two or more glycine residues or a peptide sequence with alternating glycine and serine residues, in particular the amino acid sequences Gly-Gly, Gly-Ser-Gly, and Gly-Gly-Ser-Gly-Gly. With regard to glycine-rich linkers reference is made to Witchlow M. et al., “An improved linker for single-chain Fv with reduced aggregation and enhanced proteolytic stability”, (1993) Prot. Engineering, 6:989-995.

As used herein, a “localization signal” is a signal, in particular a peptidic signal, which leads to the compartmentalization of the polypeptide carrying it to a particular part of the cell, for example an organelle or a particular topographical localization like the inner or outer face of the cell membrane. Such a localization signal can be a nuclear localization signal, a nuclear export signal, a signal that leads to targeting to the endoplasmic reticulum, the mitochondrium, the Golgi, the peroxisome the cell membrane, or even to localize sub-fractions thereof, like pre- and/or postsynaptic structures.

A “ratio change” as used herein is defined by the following formula

$$Ratio change [ \% ] = ( \frac{( \frac{IntensityYFP}{IntensityCFP} ) inCa 10 mM}{( \frac{IntensityYFP}{IntensityCFP} ) inCafree} \cdot 100 ) - 100$$

To obtain the ratio change in % of a modified polypeptide of the invention, the fluorescence emission intensities of the FRET-donor and the FRET-acceptor are measured at their respective emission maxima under suitable conditions. First, the values are determined in a calcium-free buffer solution. For example, the calcium-free buffer solution contains an aliquot of the modified polypeptide of the invention to be tested in 10 mM MOPS pH 7.5, 100 mM KCl and 20 μM EGTA. After the first measurement a solution of 1 M CaCl₂ is added to the mix to a final concentration of 10 mM CaCl₂. Then the respective emission maxima of the FRET-donor and the FRET-acceptor are measured again. The concentration of the modified polypeptide of the invention to be tested in this manner should be such that the change in FRET is readily detected. As a guideline, suitable concentrations range from 500 nM to 5 μM. Reference is made to Miyawaki, A. et al., “Fluorescent indicators for Ca²⁺ based on green fluorescent proteins and calmodulin.” (1997) Nature 388:882-887.

Kd-values of the calcium-binding polypeptides for Ca²⁺ ions can be determined as follows. Fusion polypeptides of CFP with the calcium-binding polypeptide followed by citrine are expressed by methods well known in the art, e.g. following the procedure of Example 2. The fusion polypeptides are purified following the procedure of Example 2 and stored in 300 mM NaCl, 20 mM NaPO₄-buffer pH 7.4. Kd-values are then determined by titration assays, in which the proteins are exposed to defined calcium concentrations in an aqueous buffer. To produce such defined calcium concentrations, a buffer system containing Ca²⁺ and its chelator K₂ EGTA is used. Aliquots of the protein are mixed with various ratios of two buffer solutions containing either 10 mM K₂ EGTA, 100 mM KCl and 30 mM MOPS pH 7.2 or 10 mM Ca EGTA, 100 mM KCl and 30 mM MOPS pH 7.2. The fluorescence emission intensities of the FRET-donor and the FRET-acceptor are then recorded at various concentrations of free calcium. Calcium Kd-values can be calculated by plotting the ratio of the donor and acceptor proteins' emission maximum wavelength against the concentration of free calcium on a double logarithmic scale. Thus, plotting log [Ca^{2+]}_{free} on the x-axis versus log

$$( \frac{R - R min}{R max - R} \cdot \frac{{F}_{527} min}{{F}_{527} max} )$$

on the y-axis gives an x-intercept that is the log of the proteins Kd in moles/liter.

In the formula above, R is the fluorescence intensity of the emission maximum at lower wavelength (527 nm for YFP/citrine) divided by the fluorescence intensity of the emission maximum at shorter wavelength (432 mm for CFP) at the various calcium concentrations tested. Rmin is the ratio R in a calcium-free sample, i.e. in buffer 1 only. Rmax is the ratio R in the presence of the highest chosen calcium concentration, for example at 1 mM Ca²⁺ if the ratio to buffer 1 to buffer 2 is 1:1. F₅₂₇ min is the fluorescence intensity of the emission maximum at lower wavelength (527 nm for citrine) in a calcium-free sample. F₅₂₇max is the fluorescence intensity of the emission maximum at longer wavelength (527 nm for citrine) in the presence of the highest chosen calcium concentration. Further details on the measuring method are disclosed in POZZAN T and TSIEN R Y (1989) Methods Enzymol., 172:230-244.

The “local Ca²⁺ concentration” as used herein is a change in calcium concentration, particularly a rise, which is restricted either to membrane-combined cellular organelles or to cellular structures that can handle calcium relatively independently of the remained of the cytosol, such as dendritic spines or shafts or presynaptic boutons. By “local” we also mean changes in the free calcium concentration confined to submicroscopic microenvironments in the cytosol close to a cellular membrane. By “submicroscopic” we mean areas with an extension smaller than 350 nm.

As used herein, the term “inducing a change in the calcium concentration” is any experimental regime which leads to a temporal or spatial change of the calcium distribution within a cell. In the case of studies in cell lines, cell surface receptors which are coupled to the production of an intracellular messenger such as IP3 can lead to a rise in cytosolic or mitochondrial calcium in the cell, when they are activated. An example of such surface receptors are members of the family of G-protein-coupled receptors including olfactory and taste receptors, further receptor tyrosin kinases, chemokine receptors, T-cell receptors, metabotropic amino acid receptors such as metabotropic glutamate receptors or GABAb-receptors, GPI-linked receptors of the TGF beta/GDNF-(glial-derived neurotrophic factor) receptor family. Other receptors can also directly gate calcium influx into cells, such as NMDA receptors or calcium-permeable AMPA receptors. In the case of studies with indicator organisms like transgenic C-elegans or drosophila, administration of a suitable stimulus to the organism may lead to such a calcium redistribution in certain cells which can then give an observable readout. This can, for example, be the administration of a drug to the organism, but also a stimulus with a suitable modality such as of visual, acoustic, mechanic, nociceptive or of hormonal nature. The stimulus can be, for example, cold shock, mechanical stress, osmotic shock, oxidative stress, parasites or also changes in nutrient composition in the case of transgenic plants.

A “small chemical compound” as used herein is a molecule with a molecular weight from 30 D-5 kD, preferably from 100 D-2 kD. A “small organic chemical molecule” as used herein further comprises at least one carbon atom, one hydrogen atom and one oxygen atom. Such small chemical compounds can, e.g., be provided by using available combinatorial libraries.

### DETAILED DESCRIPTION OF INVENTION

The present invention is based on the discovery that the calcium-binding protein Troponin C can form the basis for particularly powerful calcium sensors. The modified polypeptide of the invention allows the measurement of calcium fluctuations in cellular microenvironments where prior art calcium sensors like the calmodulin-based “Cameleons” have failed or have only shown poor performance. Furthermore, the Troponin C-based calcium sensors of the invention show minimal interference with the intracellular signalling pathways based on calcium and are therefore, contrary to the prior art “Cameleons”, even suitable for use in transgenic vertebrates and even mammals. Thus, the present invention relates to a modified calcium (Ca²⁺)-binding polypeptide comprising (a) a first chromophor of a donor-acceptable pair for FRET, (b) a calcium-binding polypeptide with an identity of at least 80%, preferably 85%, more preferably 90%, even more preferably 95%, and most preferably with 100% identity, to a 30 amino acid long polypeptide sequence of human Troponin C or chicken skeleton muscle Troponin C or drosophila troponin C isoform 1, and (c) a second chromophor of a donor-acceptable pair for FRET, more preferably the stretch of the calcium-binding polypeptide with this high degree of identity to human Troponin C or chicken skeletal muscle Troponin C or drosophila troponin C isoform 1 is a 35 amino acid, 40, 45, 50, 55, 60, 65, 70, or even 75 amino acid long polypeptide sequence. These polypeptides are capable of binding Ca²⁺ ions which induces a conformational change. This functionality can readily be determined as described above. Suitable chromophores are both small fluorescent molecules like, for example, the indocyanin dyes Cy3, Cy3.5, Cy5, Cy7, coumarin, fluoresceine or rhodamine, but also fluorescent polypeptides, like certain derivatives of GFP, the “green fluorescent protein”, in particular mutants of GFP with increased stability, or changed spectral characteristics, like EGFP, CFP, BFP, YFP, Cop-Green or Phi-Yellow. Other suitable fluorescent polypeptides are cFP 484 from Clavularia and zFP 538, the Zoanthus yellow fluorescent protein. As explained above, the donor chromophore and the acceptor chromophore of a donor-acceptor-pair for FRET must be chosen with regard to their spectral characteristics. As a general rule, a donor chromophore has an absorbance-maximum at lower wavelength, i.e. absorbing higher energy radiation, than an acceptor chromophore. For that reason, CFP, EGFP and YFP (citrine), all derived from Aequoria victoria, DsFP 483 from Discosoma striata., cFP 484 from Clavularia sp., AmCyan from Anemonia majano, Azami-Green from Galaxeidae sp., As499 from Anemonia sulcata and Cop-Green from Pontellina plumata. (see Tsien R. Y. “The green fluorescent protein”. Ann. Rev. Biochem. 67: 509-544 (1998); Matz M. V. et al. “Fluorescent proteins from nonbioluminescent Anthozoa species.” Nat. Biotechnol. 17: 969-973 (1999); Wiedenmann J. et al. “Cracks in the β-can: Fluorescent proteins from Anemonia Sulcata (Anthozoa, Actinaria)” Proc. Natl. Acad. Sci. 97: 14091-14096 (2000); Labas Y. A. et al. “Diversity and evolution of the green fluorescent protein family”. Proc. Natl. Acad. Sci. 99: 4256-4261 (2002). Karasawa S. et al. “A green emitting fluorescent protein from Galaxeidae coral and its monomeric version for use in fluorescent labelling. J. Biol. Chem. [epub ahead of print] (2003); Shagin D. A. et al. “GFP-like proteins as ubiquitous metazoan superfamily: evolution of functional features and structural complexity” Mol. Biol. Evol. 21(5): 841-50 (2004)) can be commonly used as donor chromophores. Examples of commonly used acceptor chromophores are YFP (citrine), DsRed from Discosoma sp., zFP 538 from Zoanthus sp., HcRed from Heteractis crispa, EqFP 611 from Entacmaea quadricolor, AsFP 595 from Anemonia sulcata, J-Red from Anthomedusae sp., and Phi-Yellow from Phialidium sp. (see Matz M. V. et al. “Fluorescent proteins from nonbioluminescent Anthozoa species.” Nat. Biotechnol. 17: 969-973 (1999); Wiedenmann J. et al. “Cracks in the β-can: Fluorescent proteins from Anemonia Sulcata (Anthozoa, Actinaria)” Proc. Natl. Acad. Sci. 97: 14091-14096 (2000); Gurskaya N. G. et al. “GFP-like chromoproteins as source of far-red fluorescent proteins” FEBS lett. 507: 16-20 (2001); Wiedenrnann J. et al. A far red fluorescent protein with fast maturation and reduced oligomerization tendency from Entacmaea quadricolor (Anthozoa, Actinaria)” Proc. Natl. Acad. Sci. 99: 11646-11651 (2002); Shagin D. A. et al. “GFP-like proteins as ubiquitous metazoan superfamily: evolution of functional features and structural complexity” Mol. Biol. Evol. 21(5): 841-50 (2004)). The example of YFP and Phi-Yellow makes clear that depending on its partner in a donor-acceptor-pair for FRET, a particular chromophore may serve as either a donor or an acceptor. For example, both YFP and Phi-Yellow can serve as an acceptor chromophore, when in combination with BFP, CFP, cFP 484, AmCyan, Cop-Green, or DsFP483, and they can function as a donor chromophore when in combination with DsRed, EqFP 611, J-Red or HcRed. By analysing the spectral characteristics of two chromophores, the skilled person can identify suitable donor-acceptor-pairs for FRET.

The three components of the modified calcium-binding polypeptide of the invention are linked together by covalent linkages. These covalent linkages between the components (a) and (b) and the components (b) and (c) can be effected by chemical crosslinking. That is, the three components can initially be independent of one another and can then be crosslinked chemically, for example by a spacer which can be selected from the group consisting of bifunctional crosslinkers, flexible amino acid linkers, like the hinge region of immunoglobulins, and homo- and heterobifunctional crosslinkers. For the present invention preferred linkers are heterobifunctional crosslinkers, for example SMPH (Pierce), sulfo-MBS, sulfo-EMCS, sulfo-GMBS, sulfo-SIAB, sulfo-SMPB, sulfo-SMCC, SVSB, SIA and other crosslinkers available, for example from the Pierce Chemical Company (Rockford, Ill., USA). Such a preferred chemical crosslinker has one functional group reactive towards amino groups and one functional group reactive towards cystine residues. The above-mentioned crosslinkers lead to formation of thioether bonds, but other classes of crosslinkers suitable in the practice of the invention are characterized by the introduction of a disulfide linkage between the polypeptides of (b) and the component (a) and/or between the polypeptide of (b) and the component of (c). It is apparent that activated conjugates of small chemical fluorophores, like FITC or like rodamine succinimidyl esters, can directly react with nucleophiles like the sulfhydryl groups of cysteins or the amino groups of lysines in the calcium-binding polypeptide of component (b) and thereby create a covalent linkage between (a) and (b) and/or (b) and (c). Amine-reactive dyes and thiol-reactive dyes can be obtained, for example from Molecular Probes Europe B V, Leyden, The Netherlands.

In a preferred embodiment, the modified calcium-binding polypeptide comprises a first chromophore (a), which is a fluorescent polypeptide capable of serving as a donor-chromophore in a donor-acceptor-pair for FRET, and a second chromophore (c), which is a fluorescent polypeptide capable of serving as an acceptor-chromophore in a donor-acceptor-pair for FRET. Preferably, the three polypeptides are part of one fusion polypeptide and the order of the three linked polypeptides starting from the N-terminus of the fusion polypeptide may be (a)-(b)-(c) or (c)-(b)-(a). It is to be understood that there may be further amino acids in the fusion polypeptide at the N— or at the C-terminus as well as between the polypeptides (a) and (b) and/or (b) and (c). In a preferred embodiment, the first chromophore (a) is selected from the group consisting of CFP, EGFP and YFP (Citrine), all derived from Aequoria victoria, Cop-Green from Pontellina plumata, Phi-Yellow from Phialidium sp., DsFP 483 from Discosoma striata, AmCyan from Anemonia majano, cFP 484 from Clavularia sp., Azami-Green from Galaxeidae sp. and As499 from Anemonia sulcata.

In another preferred embodiment, the second chromophore is selected from the group consisting of small fluorescent molecules like, for example, the indocyanin dyes Cy3, Cy3.5, Cy5, Cy7, fluoresceine or rhodamine, but also fluorescent polypeptides, like certain derivatives of GFP, the “green fluorescent protein”, in particular mutants of GFP with increased stability, or changed spectral characteristics, like EGFP, CFP, BFP or YFP. Other suitable fluorescent polypeptides are cFP 484 from Clavularia and zFP 538, the Zoanthus yellow fluorescent protein as well as Cop-Green from Pontellina plumata, and Phi-Yellow from Phialidium sp.

In another preferred embodiment, the calcium-binding polypeptide of component (b) comprises at least one calcium-binding EF-hand, and in particular comprises 2 or even 3 calcium-binding EF-hands. Most preferably, the modified calcium-binding polypeptide of the invention contains 4, 3, 2 or even only 1 EF-hand. The skilled person will appreciate that the ancestral calcium-binding site of human Troponin C, chicken skeletal muscle Troponin C or drosophila Troponin C may be genetically engineered such that its calcium-binding properties are restored.

In a preferred embodiment the calcium-binding polypeptide of the invention comprises a polypeptide sequence with at least 60% identity, more preferably at least 70%, even more preferably at least 80%, even more preferably at least 90% or most preferably 100% identity to amino acids 15-163 of chicken skeletal muscle Troponin C or amino acids 1-161 of human cardiac Troponin C or amino acids 5-154 of drosophila troponin C isoform 1. In this case, 100% identity means 100% identity over the complete 148, 161 or 149 amino acid stretch, respectively.

As mentioned previously, there can be linker sequences between component (a) and (b) and component (b) and (c) of the modified calcium-binding polypeptide of the invention. In one preferred embodiment the polypeptide of the invention therefore further comprises glycin-rich linker-peptides N-terminal or C-terminal to polypeptide (b), particularly directly neighbouring polypeptide (b) on its N-terminus or C-terminus.

In another preferred embodiment the modified calcium-binding polypeptide of the invention further comprises a localization signal, in particular a nuclear localization sequence, a nuclear export sequence, an endoplasmic reticulum localization sequence, a peroxisome localization sequence, a mitochondrial input sequence, a mitochondrial localization sequence, a cell membrane targeting sequence, and most preferably a cell membrane targeting sequence mediating localization to pre- or postsynaptic structures. It has been found that a particular advantage of the modified calcium-binding polypeptides of the invention is that they function in the context of subcellular environments where prior art calcium sensor have failed to work or have shown poor performance. The calcium sensors of the present invention are therefore particularly powerful when targeted to specific subcellular structures, like organelles or functionally distinct regions of the cell-like lamellipodia or filophodes or axons and dendrites in the case of neuronal cells. Such subcellular targeting can be accomplished with the help of particular targeting sequences. Localization to the endoplasmic reticulum can be achieved by fusing the signal peptide of calreticulin, MLLSVPLLLGLLGLAAAD to the N-terminus of a fusion polypeptide and the sequence KDEL as an ER retention motive to the C-terminus of a fusion polypeptide (discussed in Kendal et al. “Targeting aequorin to the endoplasmic reticulum of living cells.” Biochem. Biophys. Res. Commun. 189:1008-1016, (1992)). Nuclear localization can be achieved, for example, by incorporating the bipartite NLS from nucleoplasmin in an accessible region of the fusion polypeptide or, alternatively, the NLS from SV 40 large T-antigen. Most conveniently, those sequences are placed either at the N— or the C-terminus of the fusion polypeptide.

Nuclear exclusion and strict cytoplasmic localization can be mediated by incorporating a nuclear export signal into the modified calcium-binding polypeptide of the invention. Such signals are useful when the modified calcium-binding polypeptide of the invention is smaller than 60 kDa. Nuclear exclusion may not be necessary for modified calcium-binding polypeptides of the invention which are larger than 60 kDa because such polypeptides usually do not enter the cell nucleus and are therefore cytosolic at steady state. Suitable nuclear export signals are the NES from HIV Rev, the NES from PK1, AN3, MAPKK or other signal sequences obtainable from the NES base (http://www.cbs.dtu.dk/databases/NESbase/). For review of nuclear localization signals and nuclear export signals see Mattaj & Englmeier, “Nuclear cytoplasmic transport: the soluble phase” (1998), Annu. Rev. Biochem. 67:265-306.

Mitochondrial targeting can be achieved by fusing the N-terminal 12 amino acid pre-sequence of human cytochrome C oxidase subunit 4 to the N-terminus of a fusion polypeptide (for reference see Livgo, T. “Targeting of proteins to mitochondria” (2000) FEBS Letters, 476:22-26; and Hurt, E. C. et al. (1985) Embo J., 4:2061-2068). Targeting to the Golgi apparatus can be achieved by fusing the N-terminal 81 amino acids of human galactosyl transferase to the N-terminus of a fusion polypeptide and leads to targeting to the trans-cisterne of the Golgi apparatus. (For reference see Liopis J. et al. (1999) Proc. Natl. Acad. Sci. USA 95(12):6803-8.)

Suitable targeting sequences for peroxisomal targeting are PTS1 and PTS2. (For reference see Gould S. G. et al. (1987) J. Cell Biol. 105:2923-2931; and Ozurni T. et al. (1991) Biochem. Biophys. Res. Commun., 181:947-954.)

For targeting to the inner leaflet of the cell membrane, the first 20 amino terminal amino acids of GAP-43 (growth associated protein) are useful, i.e. the sequence MLCCMRRTKQVEKNDEDQKI. Alternatively, membrane targeting can be achieved by fusing the 20 most C-terminal amino acids of C-Ha-Ras to the C-terminus of a fusion polypeptide. These amino acids are KLNPPDESGTGCMSCKCVLS. (For reference see Moryoshi K. et al. (1996) Neuron, 16:255-260.)

Targeting to postsynaptic sites can be achieved by fusing the C-terminal PDZ-binding domain of the NMDA-receptor 2B subunit to the C-terminus of a fusion polypeptide. The sequence is VYEKLSSIESDV. Alternatively, the PDZ-binding domain of the inwardly rectifying potassium channel KIR 2.3 can be used as a localization when added to the C-terminus. The sequence is MQAATLPLDNISYRRESAI. (For reference see Liedhammer M. et al. (1996) J. Neurosci., 16:2157-63, and Lemaout S. et al. (2001) Proc. Natl. Acad Sci. USA, 98:10475-10480.) Other PDZ-binding domains useful for localizing indicators can be found in Hung and Sheng (2001) J. Biol. Chem., 277:5699-5702.

Presynaptic targeting can be achieved by fusing presynaptic protein such as syntaxin or synaptobrevin (VAMP-2) to the fusion polypeptides of the invention. (For reference see Bennett et al. (1992) Science, 257:255-259, and Elferink et al. (1989) J. Biol. Chem., 264:11061-4.)

A further preferred embodiment of the invention is a modified calcium-binding polypeptide of the invention which exhibits a ratio change upon calcium addition of more 30%, preferably from 50% to 200%, more preferably from 80% to 180%, even more preferably from 90% to 160%, and most preferably from 100% to 150%. Ratio change is as defined above and calcium is added to a final concentration 10 mM CaCl₂ (i.e. an appropriate volume of an 1 M aqueous solution of CaCl₂ is added to a buffer containing the polypeptide of the invention, 10 mM MOPS, pH 7.5, 100 mM KCl and 20 μM EGTA so that the final concentration is 10 mM CaCl₂. The polypeptides exhibiting such ratio changes are particularly preferred because they facilitate the measurement of calcium concentration changes within a living cell due to their low signal-to-noise ratio.

In another preferred embodiment, the modified calcium binding polypeptide of the invention has a Kd for Ca²⁺ of below 800 μM, preferably of from 50 nM to 400 μM, more preferably of from 100 mM to 100 μM, and most preferably of from 250 nNM to 35 μM. As shown in the exemplifying section, the Kd of the modified calcium-binding polypeptide of the invention for Ca²⁺ ions can be manipulated by targeted mutation of the calcium-binding EF-hands of the Troponin C-derived polypeptide. (For reference see Szczesna et al., (1996) J. Biol. Chem. 271:8381-8386, and Sorensen et al., (1995) J. Biol. Chem. 270:9770-9777). In these references the effects of mutations within the 12 amino acid loops of the EF-hand on the Kd of a calcium-binding polypeptide for calcium are explained. Thus, within certain limits, calcium-binding biosensors can be designed which have the desired affinity for calcium ions.

In a further preferred embodiment, the modified calcium-binding polypeptide of the invention is a fusion polypeptide selected from any one of the polypeptides of SEQ ID NO2, 4, 6, 8, 10, 12, 14, 16, 18, 32, 34, and 42; preferably 2, 4, 34, or 42.

In another aspect the invention provides a nucleic acid molecule comprising a nucleic acid sequence which encodes any one of the above-mentioned fusion polypeptides. In particular, a fusion polypeptide wherein the order of the three linked polypeptides starting from the N-terminus of the fusion polypeptide is (a)-(b)-(c) or (c)-(b)-(a). In a preferred embodiment the nucleic acid comprises (i) a nucleic acid sequence as defined in the SEQ IDs NO:1, 3, 5, 7, 9, 11, 13, 15, 17, 31, 33, or 41, preferably 1, 3, 33, or 41 (ii) a nucleic acid sequence which is degenerate as a result of the genetic code to the nucleic acid as defined in (i) and which encodes a polypeptide as defined in SEQ IDs NO:2, 4, 6, 8, 10, 12, 14, 16, 18, 32, 34, and 42, preferably 2, 4, 34, or 42, or a polypeptide with at least 80% identity to said polypeptides within a 30 amino acid stretch, preferably within a stretch of 45, 60 or even 75 amino acids.

A further embodiment of the invention is a recombinant expression cassette, in particular a vector, comprising a nucleic acid of the invention which is operably linked to at least one regulator sequence allowing expression of the modified protein of the invention. For example, a nucleic acid sequence encoding a modified polypeptide of the invention can be isolated and cloned into an expression vector and the vector can then be transformed into a suitable host cell for expression of a modified polypeptide of the invention. Such a vector can be a plasmid, a phagemid or a cosmid. For example, a nucleic acid molecule of the invention can be cloned in a suitable fashion into prokaryotic or eukaryotic expression vectors (Molecular Cloning: A Laboratory Manual, 3^{rd} edition, eds. Sambrook et al., CSHL Press 2001). These expression vectors comprise at least one promoter and can also comprise a signal for translation initiation and—in the case of prokaryotic expression vectors—a signal for translation termination while in the case of eukaryotic expression vectors preferably expression signals for transcriptional termination and polyadenylation are described. Examples for prokaryotic expression vectors are, for expression in Escherichia Coli, e.g. expression vectors based on promoters recognized by T7 RNA polymerase as described in U.S. Pat. No. 4,952,496, for eukaryotic expression vectors, for expression in Saccharomyces cerevisiae, e.g. the vectors G426/MET25 or P526/GAL1 (Mumberg et al. (1994), Nucl. Acids Res., 22:5767-5768), for the expression in insect cells, e.g. via bacculovirus vectors, those described by Ziccarone et al. (“Generation of recombinant bacculovirus DNA in E. coli using bacculovirus shuttle vector” (1997) Volume 13, U. Reischt et. (Totoba, N.J.: Humana Press Inc.) and for expression in mammalian cells, e.g. SW40-vectors, which are commonly known and commercially available, or the Sindbis virus expression system (Schlesinger (1993) Trans Bio Technol. 11(1):18-22) or an adenovirus expression system (Heh et al. (1998) Proc. Natl. Acad. Sci. USA 95:2509-2514). The molecular biological methods for the production of these expression vectors as well as the methods for transfecting host cells and culturing such transfecting host cells as well as the conditions for producing and obtaining the polypeptides of the invention from said transformed host cells are well known to the skilled person.

In another example, a nucleic acid molecule of the invention can be expressed in eukaryotic cells or tissue by integrating it into the host organism's genome by mechanical methods such as microinjection of DNA into oocytes or by transfection methods such as as retrovirus or lipofectin transfection of embryonic stem cells or whole embryos (Manipulating the Mouse Embryo: A Laboratory Manual, 3rd edition; Nagy et al. eds. (2002), CSHL Press, Cold Spring Harbor). DNA of the invention can be inserted in a random or a targeted manner into the context of another gene, i.e. integrated into the regulatory, 5′-, intronic, or 3′-flanking sequences of a different gene that may be endogenous or exogenous to the host organism. Suitable expression systems are for example the mouse Thy-1.2 expression cassette as described by Caroni (“Overexpression of growth-associated proteins in the neurons of adult transgenic mice” (1997) J. Neurosci. Methods 71:3-9), the CamKII promoter system (Mayford M. et al. (1996) Science, 274: 1678-1683), the GFAP promoter system (Toggas, S. M. et al. (1994) Nature 367: 188-193), the smooth muscle myosin heavy chain (smMHC) promoter (Mack C P and Owens G K (1999) Circ Res 84: 852-861), and the insulin promoter (Herrera P L et al. (1998) Mol Cell Endo 140:45-50).

In another aspect the invention relates to a host cell comprising a polypeptide, in particular a fusion polypeptide of the invention and/or a nucleic acid of the invention. Such a host cell can be a non-human cell inside or outside the animal body or a human cell outside the human body. Particularly preferred are mammalian cells like HEK cells, HELA cells, PC12 cells, CHO cells, NG108-15 cells, Jurkat cells, mouse 3T3 fibroblasts, mouse hepatoma (hepa 1C1C7 cells), mouse hepatoma (H1G1 cells), human neuroblastoma cell lines, but also established neuronal and cancer cell lines of human and animal origin available from ATCC (www.atcc.org). But host cells can also be of non-mammalian origin or even of non-vertebrate origin, like Drosophila Schneider cells, yeast cells, other fungal cells or even grampositive or gramnegative bacteria. Particularly preferred are cells within a transgenic indicator organism and also the transgenic indicator organisms comprising the host cell of the invention. The generation of transgenic flies, nematodes, zebrafish, mice and plants, for example Arabidopsis thaliana, are well established. For the generation of transgenic mice with suitable cell- or tissue-specific promoters such as the Thy-1.2 expression cassette reference is made to Hogan D. et al. (1994) “Production of transgenic mice” in Manipulating the Mouse Embryo: A Laboratory Manual—Hogan D., Constantini, F., Lacey E. eds., Cold Spring Harbor Laboratory Press, Cold Spring Harbor N.Y., pp. 217-252 and Caroni (1997) “Overexpression of growth-associated proteins in the neurons of adult transgenic mice” J. Neurosci. Methods 71:3-9. As an alternative, indicators can be expressed as transgenic mice with an inducible system; reference is made to Albanese C. et al., “Recent advances in inducible expression in transgenic mice” (2002) Semin. Cell. Dev. Biol., 13:129-41. Methods for the generation of transgenic flies, nematodes, zebrafish, and transgenic plants are also well established and exemplatory reference is made to the following documents: Rubin & Spreadling (1982) Science 218:348-53, for the generation of transgenic flies; Mellow & Fire (1995) in: Methods in Cell Biology, Volume 48, H. F. Epstein & T. C. Shakes, eds. (San Diego, Calif.: Academic Press), pp. 451-482, Higashiyima et al. (1997), Dev. Biol. 192:289-99 for the transformation of zebrafish, and Bechtold et al. (1993) C. R. Acad. Sci. [III] 316:1194-1199 for the transformation of Arabidopsis thaliana plants.

In another aspect the invention relates to a method for detecting changes of the local calcium concentration which comprises the following steps: (a) providing a cell or a subcellular membrane as faction of a cell, which cell or subcellular membranous faction comprise a calcium-binding modified polypeptide of the invention, (b) inducing a change in the local calcium concentration, and (c) measuring FRET between the donor and the acceptor chromophore of the donor-acceptor-pair of said modified calcium-binding polypeptide of the invention, wherein a change in FRET as a response to step (b) is indicative of a change in the local calcium concentration. These steps are all performed under suitable conditions. Provision of the cell in step (a) can be achieved by providing a host cell of the invention, for example a host cell transfected with an expression construct coding for a fusion polypeptide of the invention, in which, as an example, a polypeptide of the invention is expressed, e.g. as in the examples 3, 4, or 5. A subcellular membranous fraction comprising a calcium-binding polypeptide of the invention can be obtained by biochemical fractionation of the cellular constituents of, for example, above-mentioned host cell. A subcellular membranous fraction can, for example, be Golgi or ER-derived vesicles from said host cell or isolated organelles from said host cell, like pelleted nuclei or mitochondria The methods to obtain subcellular membranous fractions from, for example, cells from cell culture, are well known in the art (for reference see for example McNamee, MG (1989) Isolation and characterization of cell membranes, Biotechnique 7: 466-475 and Joost H G and Schurmann, A. (2001), Subcellular fractionation of adipocytes and 3T3 L1 cells, Meth. Mol. Biol. 155:77-82).

In a preferred embodiment, the subcellular membranous factioned is an organelle, in particular a mitochondrium, peroxisome, or a nucleus, or a membrane faction derived from a membrane-bound organelle. Particularly interesting are membrane factions derived from the cell membrane. However, in a preferred embodiment a cell, for example a cell in the context of a cell culture dish or a cell in the context of a transgenic organism, if the cell is a non-human cell, is provided in step (a). And preferably the calcium-binding polypeptide of the invention is targeted to a specific subcellular localization within said cell, and most preferably is targeted to the inner surface of the cell membrane.

As explained above, a change in the local calcium concentration can be induced by various stimuli, like the administration of extracellular stimuli. In a preferred embodiment step (b) is effected by the administration of an extracellular stimulus, and in particular by the addition of a small chemical compound or a polypeptide to the extracellular side of the host cell. Step (b) can also be effected by extracellular or intracellular electrical stimulation of the host cell, for example with microelectrodes. In addition, step (b) can be induced in a whole organism using various sensory stimuli such as visual, olfactory and auditory stimuli. If changes of the local calcium concentration are to be measured in a cell in the context of a cell culture dish, then it is preferred that the fusion polypeptides of the invention are co-expressed together with a receptor protein or ion channel protein of interest whose activation can be read out in the form of a calcium signal. Such receptors can be receptors coupled to the production of an intracellular messenger such as IP3 that leads to a rise in cytosolic or mitochondrial calcium in the cell when the receptor or ion channel is stimulated, for example, members of the family of G-protein coupled receptors including olfactory and taste receptors, receptor tyrosine kinases, chemokine receptors, T-cell receptors, metabotropic amino acid receptors such as metabotropic glutameic receptors or Gabab-receptors, GPI-linked receptors of the TGFbeta/GDNF-(glial-derived neurotrophic factor) receptor family. Receptors can also be directly gating calcium influx into transfector cells such as NMDA receptors or calcium-permeable AMPA receptors. Also interesting are calcium channels that are gated by membrane potential under physiological conditions, such as L-type, P/Q-type and N-type calcium channels. After co-expression of the calcium sensors of the invention and such an receptor or channel has been achieved, in the next step (b) an agonist or antagonist of said receptor or channel is provided to the co-transfected host cell, and then in step (c) the change in the local calcium concentration can be read out on a microscope stage by exciting the donor chromophor at a suitable wavelength using a suitable light source such as Xenon Arc Lamp, a monochromator or a laser light source, suitable dichroic mirrors and excitation filters and emission filters of suitable bandwith to extract information on the donor and acceptor emission, finally by recording the signals on a CCD (charge-coupled device) camera or a photomultiplier tube.

If the cell is provided in the context of an indicator organism, then the method is performed by expressing the fusion polypeptide of the invention in a transgenic organism in a cell or tissue type of interest with the help of suitable cell-type and developmental-stage-specific, constitutive or inducible promoters. As the next step (b), a suitable stimulus is provided to the organism, for example a drug is administered to the organism or alternatively a stimulus of suitable modality is provided to the organism, such as an electrical, sensory, visual, acoustic, mechanic, nociceptive or hormonal stimulus. This stimulus elicits a calcium signal which can be detected when the fusion polypeptide of the invention is expressed in tissues of interest within the transgenic animal, such as the nervous system or in intestinal organs. The stimulus can be, for example, a visual, auditory, or olfactory signal, electric current, cold shock, mechanical stress, osmotic shock or oxidative stress, parasites or changes in nutrient composition. The change of the local calcium concentration can then be read out by microscopy of the cell or tissue of interest, as indicted above. In addition, a tissue preparation such as an acute brain slice can be obtained from the transgenic organism and stimulated by a variety of pharmacological as well as electrophysiological stimuli.

In another aspect the invention provides a method for the detection of the binding of a small chemical compound or a polypeptide to a calcium-binding polypeptide with an identity of at least 80% to a 30 amino acid long polypeptide sequence of human Troponin C or chicken skeletal muscle Troponin C or drosophila troponin C isoform 1. This method comprises the steps of (a), providing a calcium-binding polypeptide of the invention, (b) adding a small chemical compound to be tested for binding or a polypeptide to be tested for binding, and (c) determining the degree of binding by measuring FRET between the donor- and the acceptor-chromophor of the donor-acceptor-pair of said polypeptide under suitable conditions. In a preferred embodiment the calcium-binding polypeptide provided in step (a) is human cardiac muscle Troponin C or a polypeptide derived from human cardiac muscle Troponin C and, in particular, is SEQ ID NO:4. This method is useful to identify small chemical compounds or polypeptides of clinical interest, in particular as in certain clinical settings, such as congenital heart failure, cardiomyopathy or other myocardial diseases such as induced by diabetes leading to reduced performance of the human heart. The method is also useful in identifying compounds that strengthen or weaken skeletal muscle contractive force. Such compounds that strengthen skeletal muscle function can be beneficial therapeutics in diseases leading to muscle degeneration, such as muscular dystrophies as for example Duchenne muscular dystrophy, or spinal muscular atrophy. Compounds that weaken skeletal muscle contraction may find its use in conditions that lead to excessive muscle convulsions, as for example in Tetanus. Small chemical molecules or polypeptides which help to improve the calcium-binding properties of human cardiac muscle Troponin C or human skeletal muscle troponin C have the potential of being suitable pharmaceuticals for the treatment of the above-mentioned diseases. In a preferred embodiment the small chemical compounds or the polypeptides identified by the above-mentioned screening method are formulated into a pharmaceutical composition which can be used for the treatment of the above-mentioned disease.

In another aspect the invention relates to the use of a modified calcium-binding polypeptide of the invention for the detection of changes in the local calcium concentration within a cell, and in particular for the detection of calcium changes occurring close to a cellular membrane. In one aspect this can be for diagnostic purposes in a subject, e.g. a human patient. Also the modified calcium-binding polypeptide of the invention can be used for the detection of changes in the local calcium concentration within a cell of a transgenic animal of the invention, like a transgenic mouse, or preferably a non-mammalian transgenic animal, like transgenic bakers yeast, C. elegans, D. melanogaster or zebrafish. In another aspect this use is not contemplated to be practiced on a human or animal body, but relates to an ex vivo use, in particular an in vitro use, e.g. in cell lines or in primary cells in cell culture.

In a preferred embodiment such modified calcium-binding polypeptides are used which comprise a localization signal, in particular a nuclear localization signal, a nuclear export signal, an endoplasmic reticulum localization signal, a peroxisome localization signal, a mitochondrial input signal, a cell membrane targeting signal, or a cell membrane targeting signal mediating localization to pre- or postsynaptic structures. Most preferably, such modified calcium-binding polypeptides are used which comprise a cell membrane targeting signal, and in particular a cell targeting signal mediating localization to the cell membrane of pre- or postsynaptic structures. It is desirable that the modified calcium-binding polypeptide is a genetically encoded fusion polypeptide of the invention.

### DESCRIPTION OF THE FIGURES

FIG. 1: Schematic representation of FRET occurring in ratiometric indicators based on troponin C variants.

FIG. 2: Summary of basic constructs and evaluation of their function. csTnC, chicken skeletal muscle troponin C. csTnC-N90, the N-terminal lobe of chicken skeletal troponin C (amino acids 1-90). csTnC-EFn, the individual EF hands 1-4 of chicken skeletal muscle troponin C. csTnI, chicken skeletal muscle troponin I. csTnI 1-48, csTnI 95-133, csTnI 116-135, various short peptides derived from chicken skeletal muscle troponin I consisting of the indicated amino acid residues. csTnC-L15, truncated chicken skeletal muscle troponin C in which the N-terminal amino acid residues 1-14 are deleted, which makes the protein start at leucin 15. The whole indicator construct was named TN-L15 (SEQ ID NO. 1 and 2). csTnC-L15 D107A, csTnC-L15 carrying the mutation D107A. The whole indicator construct was named TN-L15 D107A (SEQ ID NO. 5, 6). csTnC-L15-N90, N-terminal lobe of chicken skeletal muscle troponin C consisting of amino acid residues 15-90. hcardTnC, human cardiac muscle troponin C. The whole indicator construct is referred to as TN-humTnC (SEQ ID NO. 3, 4). hcardTnC1-135, human cardiac muscle troponin C lacking the last EF hand domain. hcardTnC-L12, human cardiac muscle troponin C in which the N-terminal amino acid residues 1-11 are deleted, analogous to csTnC-L15. L, linker: either GG, GSG or GGSGG.

FIG. 3: Effect of calcium binding on the emission spectrum of two indicator constructs; A: TN-L15. B: TN-humTnC. The emission spectra of the two constructs are depicted at zero (dashed line, —Ca²⁺) and saturating (solid line, +Ca²⁺) calcium levels.

FIG. 4: Calcium affinities (A) and pH-sensitivities (B) of selected indicator proteins. A: TN-humnTnC (open diamonds), TN-L15 (filled squares), TN-L15 D107A (open circles), TN-L15 E42Q/E78Q (filled circles). B: Emission ratio of TN-15 in the presence (circles) and absence (squares) of calcium at various pH values.

FIG. 5: Calcium dissociation from selected purified indicator proteins.

FIG. 6: Function of TN-L15 within live cells. A: HEK 293 cells displaying cytosolic localization and different expression levels (cell 1 and 2) of TN-L15. B: Ratio traces of the two cells depicted in A. Responses to stimulation with 100 μM carbachol and treatment with 1 μM ionomycin at high calcium to obtain Rmax and at 100 μM EGTA to obtain Rmin are shown. C: Corresponding intensity traces of CFP and Citrine emission for the ratios in B, showing individually the traces of the higher expressing cell 1 and the dimmer expressing cell 2.

FIG. 7: Function of TN-humTnC in live cells. A: Cytosolic expression of TN-humTnC in HEK293 cells. B: Imaging trace showing the 527/476 nm emission ratio after stimulation with 100 μM carbachol.

FIG. 8: Function of TN-L15 in live primary hippocampal neurons. A. Primary hippocampal neuron transfected with TN-L15: B: Imaging trace of the neuron shown in A.

FIG. 9: Targeting TN-L15 to the plasma membrane of live cells. A scheme of the construct is depicted. A: 293 cells expressing TN-L15-Ras. The arrow points at the cell whose trace is shown in B. B: TN-L15-Ras readily reports agonist-induced calcium oscillations in 293 cells. The indicator has the same dynamic range as when expressed in the cytosol. C: Primary hippocampal neuron expressing TN-L15-Ras and corresponding imaging trace (D).

FIG. 10: Comparison of fusions of TN-L15 and Yellow Cameleon 2.3 (YC2.3) to the presynaptic protein Synaptobrevin. A: Imaging trace of TN-L15-Synaptobrevin expressed in 293 cells. B: Imaging trace of YC2.3-Synaptobrevin.

FIG. 11: Comparison of membrane-targeting of TnL-15 and Yellow Cameleon 2.1 (YC2.1) using the membrane targeting sequence of GAP43. A: Imaging trace with GAP43-TN-L15 in 293 cells. B: Imaging trace with GAP43-YC2.1 in 293 cells. Note the poor performance of GAP43-YC2.1.

FIG. 12: Emission spectra of the two indicator constructs TN-TPC1 and TN-TPC1-L5 containing the drosophila troponin C isoform 1, before and after binding of calcium. Dashed line: zero calcium level, solid line: calcium saturation. A ratio change of over 150% could be observed with both constructs.

FIG. 13: In this indicator version, amino acids 15-163 of chicken skeletal muscle troponin C were fused between the chromophores Cop-Green and Phi-Yellow instead of CFP and YFP (Citrine) as FRET donor and acceptor. The figure shows the emission spectra before and after calcium binding. Dashed line: zero calcium level, solid line: calcium saturation

FIG. 14: A: Schematic drawing of the mouse Thy-1.2 expression cassette containing indicator construct TN-L15. The Thy-1.2 system drives constitutive postnatal transgene expression mainly confined to neurons; numbered boxes indicate untranslated exon sequences of the Thy1.2-gene (Caroni P., J Neuroscience Methods 71 (1997) 3-9).

B-C: Anti-GFP antibody staining showing expression patterns in hippocampus (B), and acute slice showing single neurons expressing TN-L15 in the cerebellar cortex (C) from an adult mouse (6 weeks) of mouse line Thy1.2-TN-L15-B, exemplifying the distribution of expression in the brain.

D-E: Calcium imaging trials in organotypic slice cultures, prepared from hippocampi of Thy1.2-TN-L15-B-mice at P4 and imaged after 2 weeks in culture. D: 535 nm fluorescence emission of a hippocampal slice preparation; cells are 40× magnified and excited at 432 nm. E: YFP/CFP ratio traces presumably reflecting the influx of calcium into the cells after the addition of 50 mM KCl to the slice preparation shown in D. A YFP/CFP ratio change of about 20% is visible after stimulation.

### DESCRIPTION OF THE SEQUENCES

SEQ ID NO: 1 is the DNA-sequence of TN-L15; a fusion construct of CFP, chicken skeletal muscle troponin C amino acids 15-163, and Citrine.

SEQ ID NO: 2 is the protein sequence of the construct of SEQ ID NO: 1

SEQ ID NO: 3 is the DNA-sequence of TN-humTnC; a fusion construct of CFP, human cardiac muscle troponin C, and Citrine.

SEQ ID NO: 4 is the protein sequence of the construct of SEQ ID NO: 3

SEQ ID NO: 5 is the DNA sequence of TN-L15 D107A; a fusion construct of CFP, chicken skeletal muscle troponin C amino acids 15-163, and Citrine. The third EF-hand of the troponin C is inactivated by the single amino acid exchange D107A

SEQ ID NO: 6 is the protein-sequence of the construct of SEQ ID NO: 5

SEQ ID NO: 7 is the DNA sequence of a fusion construct of CFP, chicken skeletal muscle troponin C, and Citrine

SEQ ID NO: 8 is the protein sequence of the construct of SEQ ID NO: 7

SEQ ID NO: 9 is the DNA sequence of a fusion construct of CFP, the second EF-hand of chicken skeletal muscle troponin C (amino acids 51-91), and Citrine

SEQ ID NO: 10 is the protein sequence of the construct of SEQ ID NO: 9

SEQ ID NO: 11 is the DNA sequence of a fusion construct of CFP, chicken skeletal muscle troponin C, a Gly-Gly linker, chicken skeletal muscle troponin I, and Citrine

SEQ ID NO: 12 is the protein sequence of the construct of SEQ ID NO: 11

SEQ ID NO: 13 is the DNA sequence of a fusion construct of CFP, chicken skeletal muscle troponin I amino acids 116-135, a Gly-Gly linker, chicken skeletal muscle troponin C, and Citrine

SEQ ID NO: 14 is the protein sequence of the construct of SEQ ID NO: 13

SEQ ID NO: 15 is the DNA sequence of a fusion construct of CFP, chicken skeletal muscle troponin I amino acids 95-131, a Gly-Ser-Gly linker, chicken skeletal muscle troponin C amino acids 1-91, and Citrine

SEQ ID NO: 16 is the protein sequence of the construct of SEQ ID NO: 15

SEQ ID NO: 17 is the DNA sequence of TN-L12; a fusion construct of CFP, human cardiac muscle troponin C amino acids 12-161, and Citrine

SEQ ID NO: 18 is the protein sequence of the construct of SEQ ID NO: 17

SEQ ID NO: 19 is the DNA sequence of human cardiac muscle troponin C

SEQ ID NO: 20 is the protein sequence of SEQ ID NO: 19

SEQ ID NO: 21 is the DNA sequence of human cardiac muscle troponin I

SEQ ID NO: 22 is the protein sequence of SEQ ID NO: 21

SEQ ID NO: 23 is the DNA sequence of human skeletal muscle troponin C

SEQ ID NO: 24 is the protein sequence of SEQ ID NO: 23

SEQ ID NO: 25 is the DNA sequence of chicken skeletal muscle troponin C

SEQ ID NO: 26 is the protein sequence of SEQ ID NO: 25

SEQ ID NO: 27 is the DNA sequence of chicken fast skeletal muscle troponin I

SEQ ID NO: 28 is the protein sequence of SEQ ID NO: 27

SEQ ID NO: 29 is the DNA sequence of chicken cardiac muscle troponin C

SEQ ID NO: 30 is the protein sequence of SEQ ID NO: 29

SEQ ID NO: 31 is the DNA sequence of TN-TPC1; a fusion construct of CFP, drosophila troponin C isoform 1, and Citrine

SEQ ID NO: 32 is the protein sequence of the construct of SEQ ID NO: 31

SEQ ID NO: 33 is the DNA sequence of TN-TPC1-L5; a fusion construct of CFP, drosophila troponin C isoform 1 amino acids 5-154, and Citrine

SEQ ID NO: 34 is the protein sequence of the construct of SEQ ID NO: 33

SEQ ID NO: 35 is the DNA sequence of drosophila troponin C isoform I

SEQ ID NO: 36 is the protein sequence of the construct of SEQ ID NO: 35

SEQ ID NO: 37 is the DNA sequence of drosophila troponin C isoform 2

SEQ ID NO: 38 is the protein sequence of the construct of SEQ ID NO: 37

SEQ ID NO: 39 is the DNA sequence of drosophila troponin C isoform 3

SEQ ID NO: 40 is the protein sequence of the construct of SEQ ID NO: 39

SEQ ID NO: 41 is the DNA sequence of Cop-Li5-Phi; a fusion construct of Cop-Green, chicken skeletal muscle troponin C amino acids 15-163, and Phi-Yellow

SEQ ID NO: 42 is the protein sequence of the construct of SEQ ID NO: 41

### EXEMPLIFYING SECTION

The following examples are meant to further illustrate, but not limit, the invention. The examples comprise technical features and it will be appreciated that the invention relates also to combinations of the technical features presented in this exemplifying section.

### Example 1

### Gene Construction

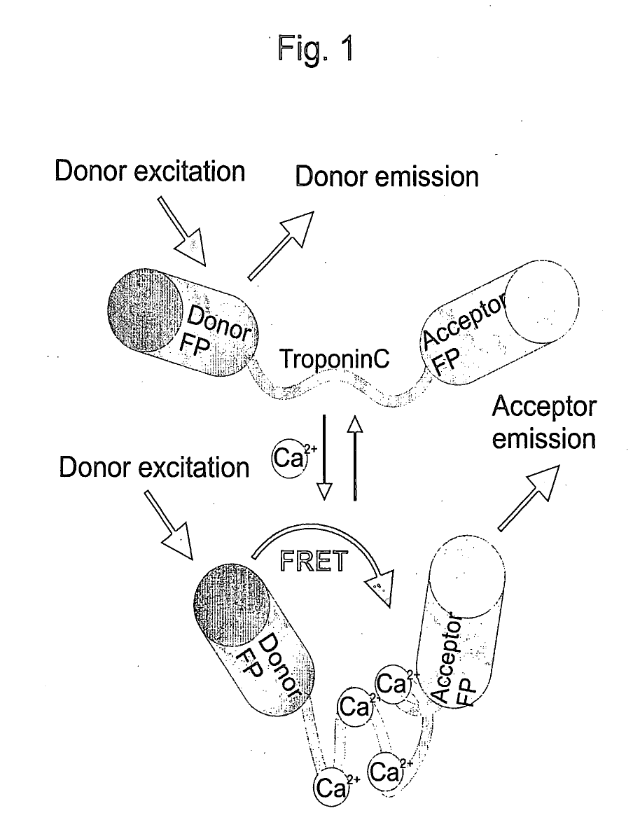

Full length and truncated troponin C domains were obtained by PCR from the cDNA of chicken skeletal muscle troponin C (csTnC) and drosophila troponin C isoform 1 (TnC41C) using a sense primer containing an SphI site at the 5′ end and a reverse primer containing a SacI site at the 3′ end. Likewise, full length and truncated domains of human cardiac muscle troponin C (hcardTnC) were obtained from a cDNA sequence from which the intrinsic SacI site had to be removed first by oligonucleotid-directed mutagenesis, resulting in a silent mutation of the Glu135 codon (GAG to GAA). All troponin C DNA fragments were inserted between CFP and Citrine in the bacterial expression vector pRSETB (Invitrogen) carrying a CFP with an SphI site at the 3′ end and a Citrine with a SacI site at the 5′ end. A schematic representation of FRET occurring in ratiometric indicators based on troponin C variants is shown in FIG. 1. Calcium binding to the troponin C domain leads to a conformational change in the protein, thereby enhancing the fluorescence resonance energy transfer (FRET) from the donor to the acceptor fluorescent protein (FIG. 1). First constructs with simple insertion of the full-length gene yielded an indicator of moderate performance. We then tested a series of mutations and deletions at the linker regions. We went through a series of optimizations in which individual amino acids at the linking sequences close to the GFPs were exchanged or deleted. Overall, more than 70 different constructs were made, the proteins purified and tested individually for their calcium sensitivity. In addition to the full length sequences of hcardTnC, TnC41C, and csTnC and versions thereof with truncations and modified linkers, shorter csTnC domains were engineered in which only specific structural elements of the protein were used individually such as the N-terminal regulatory lobe (amino acids 1-90, termed TnC-N90) of csTnC alone or individual EF-hands of csTnC.

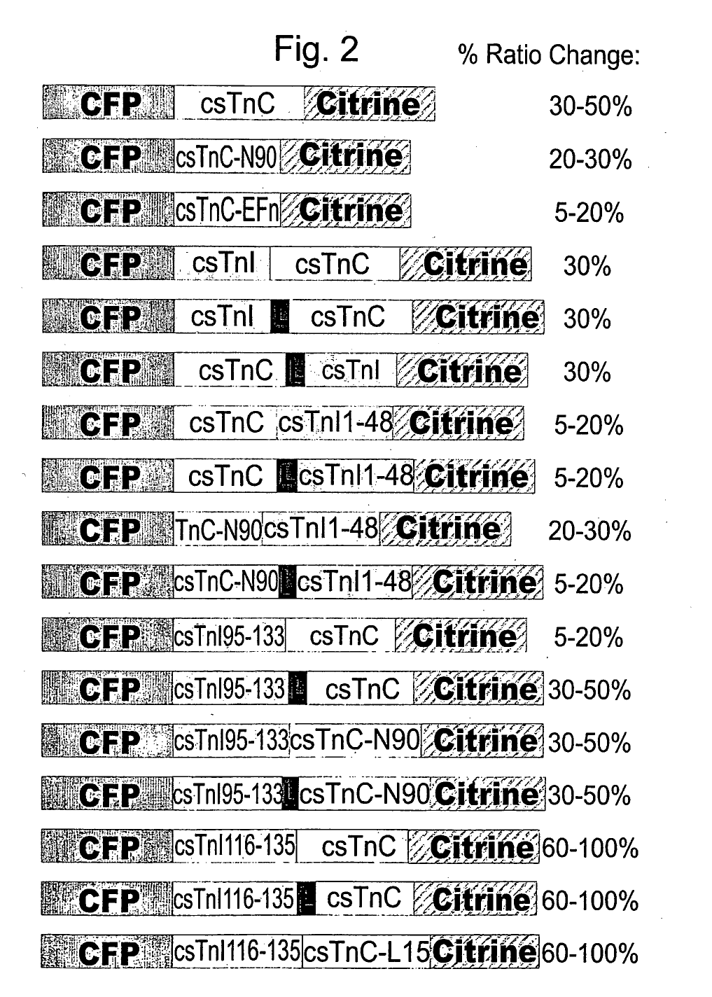

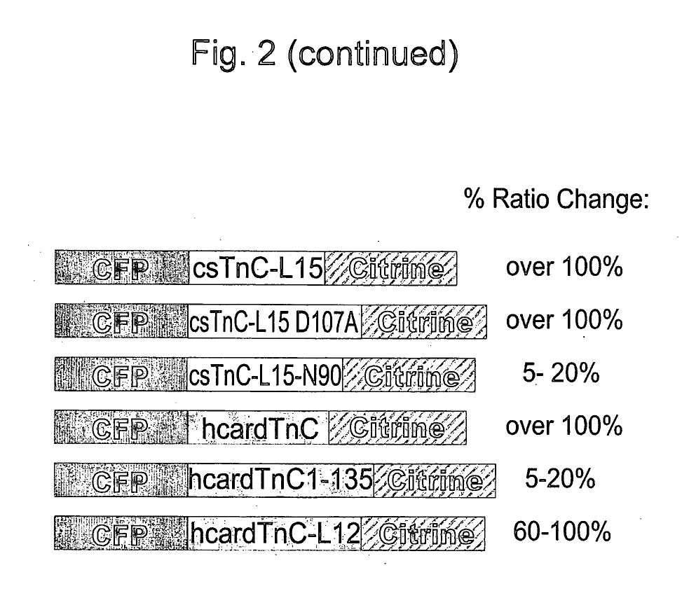

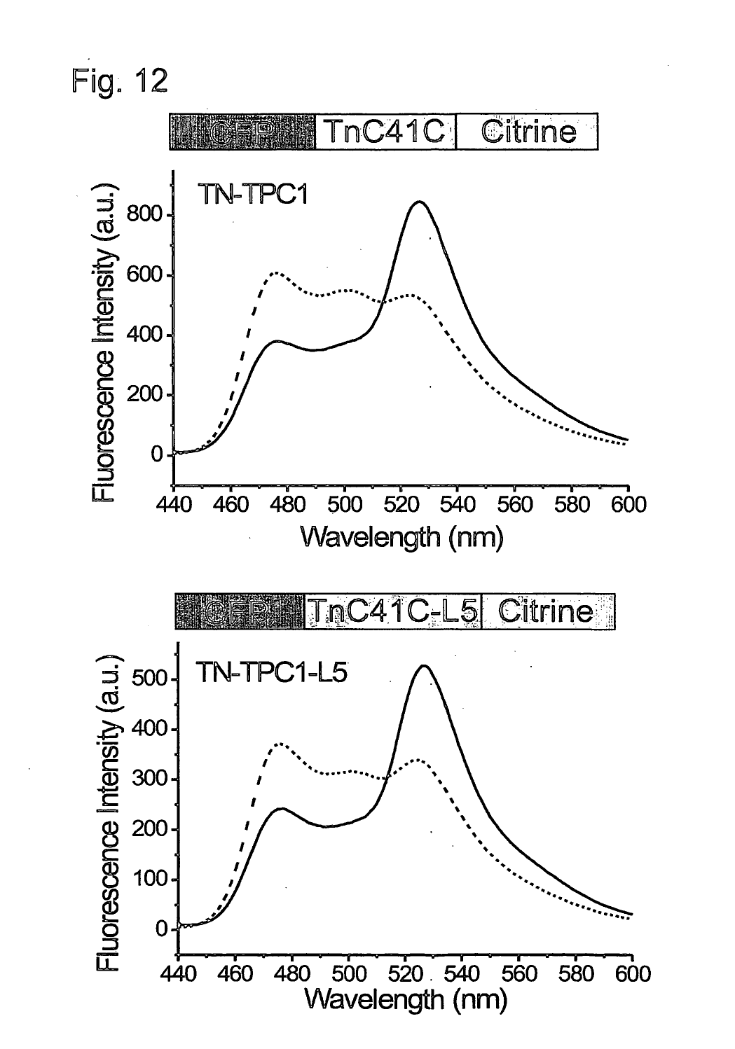

A summary of basic constructs and evaluation of their function can be seen in FIGS. 2 and 12. Only constructs with moderate to good performance are listed. Performance was evaluated as the maximal % change in the 527/476 nm emission ratio from zero calcium levels to calcium saturation. The best performing constructs giving more than 100% maximal ratio change were selected for further analysis. These constructs were named TN-humTnC for an indicator using the human cardiac skeletal muscle troponin C (hcardTnC) as calcium binding moiety (SEQ. ID 3, 4), TN-L15 for an indicator using the chicken skeletal muscle troponin C (csTnC-L15), amino acids 15-163, as calcium binding moiety (SEQ. ID 1,2), and TN-TPC1-L5 for an indicator with drosophila troponin C isoform 1 (TnC41C), amino acids 5-154, as calcium binding moiety (SEQ. ID 33, 34). Since TnI, like TnI from chicken, for example, the chicken fast skeletal muscle TnI isoform (csTnI) with the Swissprot Accession Number P02644, is known to form a complex with csTnC in vivo and some of these interactions are modified by calcium, peptide sequences of csTnI considered to be responsible for binding to the N- and C-terminal csTnC domains were selected according to the literature. csTnI fusions with csTnC were created by amplifying domains of chicken skeletal muscle TnI cDNA with sense and reverse primers containing both either an SphI site or a SacI site. The resulting csTnI DNA fragments carrying either a SphI site or a SacI site at both ends could then be cloned into the existing SphI or SacI sites in the troponin C indicator fusion constructs.

To alter calcium affinities of single EF-hands of chicken skeletal muscle troponin C, point mutations were introduced into the gene sequence by site-directed mutagenesis using the primer extension method (QuickChange, Stratagene). For protein expression in mammalian cells, an optimized Kozak consensus sequence (GCC GCC ACC ATG G) was introduced by PCR at the 5′ end of CFP; the entire indicator fragments obtained by BamHI/EcoRI restriction of the pRSETB constructs were then subcloned into the mammalian expression vector pcDNA3 (Invitrogen). Membrane targeting of indicator proteins was achieved by extending the indicator DNA sequences with a sequence encoding a membrane localization signal by PCR. In particular, the 20 amino acid sequence KLNPPDESGPGCMSCKCVLS of the c-Ha-Ras membrane-anchoring signal was fused at the 3′ end of the indicator sequences, and the 20 amino acid sequence MGCCMRRTKQVEKNDEDQKI of the GAP43 membrane-anchoring signal was fused at the 5′ end. See Moriyoshi K., et al., “Labeling Neural Cells Using Adenoviral Gene Transfer of Membrane-Targeted GFP.” Neuron 16, 255-260 (1996).

Fusions of TN-L15 (SEQ ID No: 1) or YC3.1 to Synaptobrevin were made by amplifying Synaptobrevin by PCR, thus introducing a Kpn1-Site within a GGTGGS linker to its 5′-end. Simultaneously, a Kpn1-site was introduced at the 3′ end of csTnL-15 or YC3.1, respectively. The stop codon was thereby deleted. DNA fragments coding for thus modified Synaptobrevin and TN-L15 or YC3.1 were ligated together into an expression plasmid.

For the construction of the non-Aequoria victoria-FP indicator version Cop-L15-Phi, DNA sequences of Cop-Green (Copepoda-GFP ppluGFP2) and Phi-Yellow (Phialidium-YFP) were obtained by PCR from cDNA-containing plasmids (both Evrogen). The sense primer used for the amplification of the Cop-Green insert introduced a BamHI restriction site and the Kozak sequence GCC GCC ACC ATG GCC at the 5′ end of the Cop-Green sequence, thereby adding the new amino acids Met and Gly to the N-terminus of the polypeptide chain. The antisense primer inserted a SphI restriction site at the 3′ end of the Cop Green sequence and deleted the original stop codon. The Phi-Yellow insert was amplified with a primer pair that introduced a SacI site at its 5′ end and a EcoRI site at its 3′ end. For the creation of the indicator construct Cop-L15-Phi, a chicken skeletal muscle troponin C (csTnC-L15) fragment containing amino acids 15-163 with a SphI site at the 5′ end and a SacI site at the 3′ end was ligated together with the Cop-Green and Phi-Yellow inserts into the expression vector pRSETB (Invitrogen). This resulted in the fusion protein Cop-L15-Phi with the FRET donor Cop-Green at the N-terminus, csTnC-L15 as calcium binding domain in the middle, and Phi-Yellow as FRET acceptor at the C-terminus.

### Example 2

### Protein Expression, In Vitro Spectroscopy and Titrations

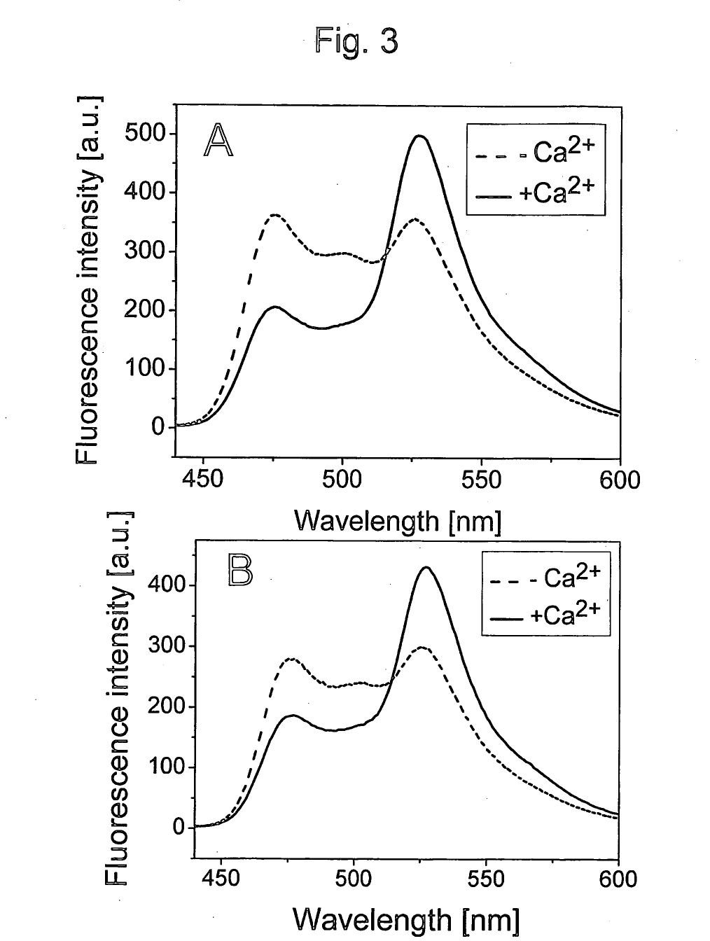

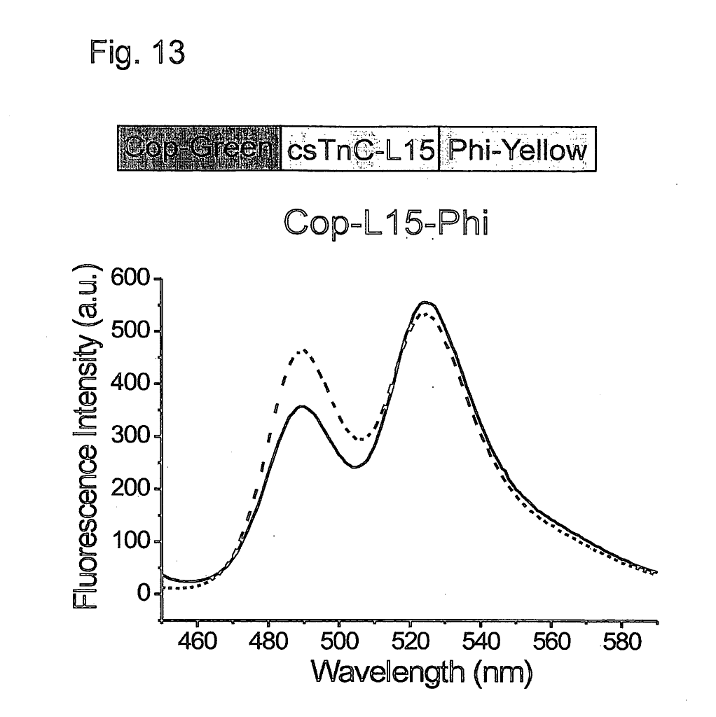

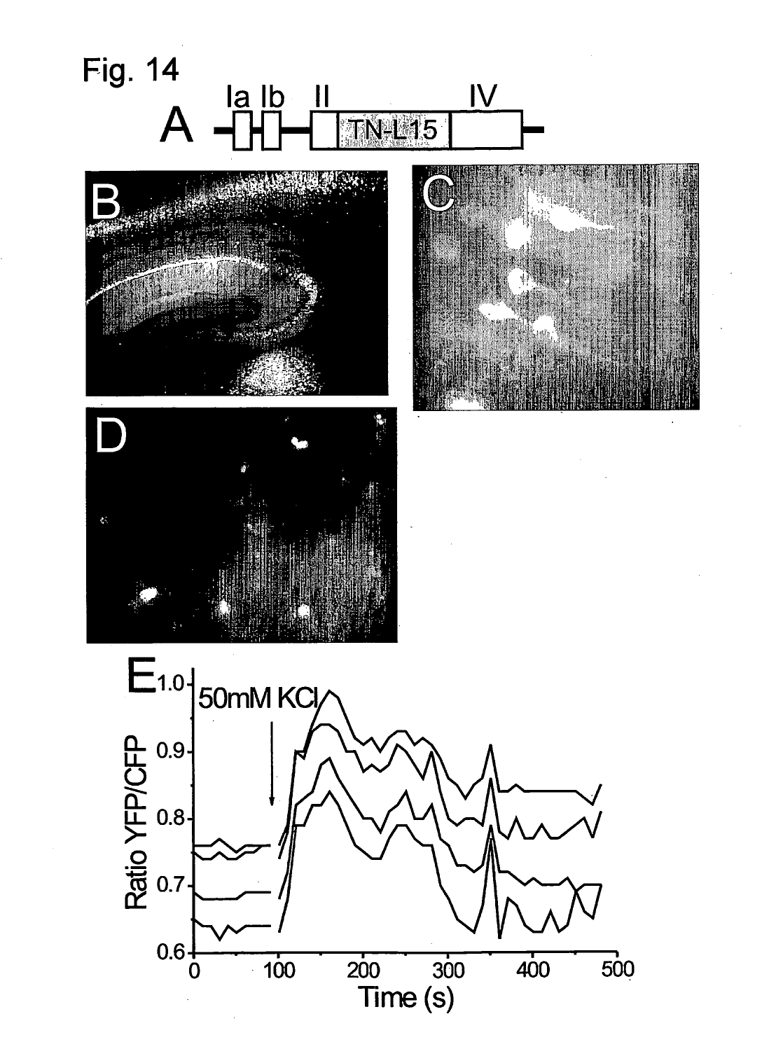

Proteins were expressed in bacteria using the T7 expression system in combination with the pRSETB plasmid carrying the fusion protein. Since the pRSETB plasmid also furnishes the fusion protein with an N-terminal polyhistidine tag, proteins could be purified from cleared cell lysates on nickel-chelate columns. Purified proteins were then subjected to in vitro fluorescence measurements in a Cary Eclipse fluorometer (Varian) equipped with a stopped flow RX2000 rapid kinetics accessory unit for kinetic measurements (Applied Photophysics). To obtain the percent ratio change of a protein, the fluorescence emission intensities of the FRET donor and the acceptor were measured at their respective emission maxima. Values were determined at zero calcium levels or at calcium saturation for each indicator. The Ca-free buffer contained an aliquot of the protein in 10 mM MOPS pH 7.5, 100 mM KCl, and 20 μM EGTA. In the second step, a solution of IM CaCl₂ was added to the mix to a final concentration of 10 mM CaCl₂. The effect of calcium binding on the emission spectrum of five indicator constructs are shown in FIGS. 3, 12 and 13. FIG. 3A shows the emission spectrum of TN-L15, a fusion protein of amino acids 15-163 of chicken skeletal muscle troponin C (csTnC) as calcium binding moiety sandwiched between CFP and Citrine. Likewise, FIG. 3B shows the emission spectrum of TN-humTnC, a fusion protein of amino acids 1-161 of human cardiac muscle troponin C (hcardTnC) as calcium binding polypeptide sandwiched between CFP and Citrine. The emission spectra of the two constructs are depicted at zero (dashed line, —Ca²⁺) and saturating (solid line, +Ca²⁺) calcium levels. The change of the emission ratio upon Ca²⁺ binding is 140% for TN-L15 and 120% for TN-humTnC. FIG. 12 shows the emission spectra of TN-TPC1 and TN-TPC1-L5, two indicators that carry the drosophila troponin C version TnC41C in a full-length and a truncated form between CFP and YFP. The change of the emission ratio after Ca²⁺ binding is 150% for TN-TPC1 and 160% for TN-TPC1-L5. To obtain the percent ratio change of a protein, the fluorescence emission intensities of the FRET donor and the acceptor were measured at their respective emission maxima. Values were determined at zero calcium levels or at calcium saturation for each indicator. The Ca-free buffer contained an aliquot of the protein in 10 mM MOPS pH 7.5, 100 mM KCl, and 20 μM EGTA. In the second step, a solution of IM CaCl₂ was added to the mix to a final concentration of 10 mM CaCl₂. The C-terminal domain of TnC is known to have two high-affinity calcium binding sites that also bind magnesium. The N-terminal lobe binds calcium specifically with a somewhat lower affinity. In agreement with this, addition of 1 mM magnesium reduced the maximal dynamic range of TN-L15 and TN-humTnC obtainable by addition of calcium from 140% to 100% and 120% to 70%, respectively.

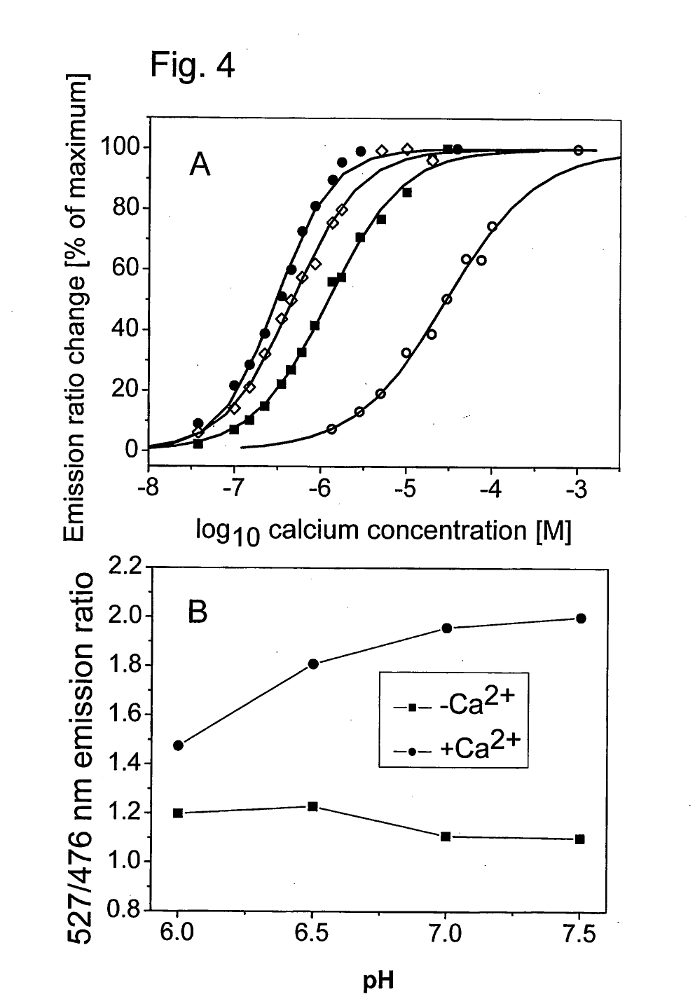

Calcium titrations were done in a buffer system containing Ca²⁺ and K₂EGTA in various ratios such as to obtain defined concentrations of free Ca²⁺. Thus, by mixing aliquots of the indicator protein with various ratios of two buffer solutions containing either 10 mM K₂EGTA, 100 mM KCl and 30 mM MOPS pH 7.2, or 10 mM CaEGTA, 100 mM KCl and 30 mM MOPS pH 7.2, the fluorescence emission intensities of the FRET donor and the acceptor could be recorded at various concentrations of free calcium. Magnesium was added to the buffers when necessary. Calcium Kd values were calculated by plotting the ratio of the donor and acceptor protein's emission maximum wavelengths against the concentration of free calcium on a double logarithmic scale. See Grynkiewicz G., et al. “A New Generation of Ca²⁺ Indicators with Greatly Improved Fluorescence Properties.” J. Biol. Chem. 260, 3440-3450 (1985). Magnesium titrations were done in 1 mM MOPS pH 7.0, 100 mM KCl and varying amounts of MgCl₂. Calcium affinities and pH-sensitivities of selected indicator proteins are depicted in FIG. 4. FIG. 4A shows the determination of calcium K_{d} values of selected constructs by Ca²⁺ titrations in the presence of 1 mM free Mg²⁺. Emission ratio changes were normalized to the values at full calcium saturation, and curve fits correspond to the apparent calcium K_{d} values given in the text. Calcium titrations resulted in response curves with apparent dissociation constants of 470 nM for TN-humcTnC (open diamonds) and 1.2 μM for TN-L15 (filled squares). K_{d}s for magnesium binding were 2.2 mM and 0.5 mM for TN-L15 and TN-humTnC, respectively. Site-directed mutagenesis has been used extensively to study ligand binding properties and conformational change within troponin C. We therefore inactivated individual EF-hands systematically by exchanging crucial aspartate or glutarnate residues within the binding loops with either alanine or glutamine. The mutation D107A, by which the third, C-terminal EF-hand was inactivated within TN-L15, resulted in an indicator with reduced calcium affinity. The apparent calcium K_{d} of this construct was determined to be 29 μM (open circles). As a consequence, the response curve in calcium titrations was significantly shifted to the right, as seen in FIG. 4A. Therefore, this mutant appears to be more suitable for measuring larger changes in calcium that can be encountered for example when targeting indicators to synaptic sites or in close vicinity to channels. For comparison, however, inactivating both N-terminal sites by the double mutation E42Q/E78Q yielded a protein that left only the C-terminal high-affinity components intact, resulting in a K_{d} for calcium of 300 nM (FIG. 4A, filled circles). In FIG. 4B, we investigated to what extent pH changes affected the ratios of TN-L15 obtained at zero calcium (50 μM BAPTA, filled square, —Ca²⁺) or calcium saturation (10 mM Ca²⁺, filled circle, +Ca²⁺). As expected, ratios were dependent on pH. Ratios started to drop beginning below pH 6.8 reflecting the pH-properties of Citrine and CFP. In the physiological range of cytosolic pH fluctuations between pH 6.8-7.3 the ratios were, however, remarkably stable. pH-resistance of our probes is a clear advantage over recent non-ratiometric probes based on calmodulin and a single GFP as fluorophore, as these probes are intrinsically sensitive to pH changes and therefore artifact-prone even when expressed in the cytosol.

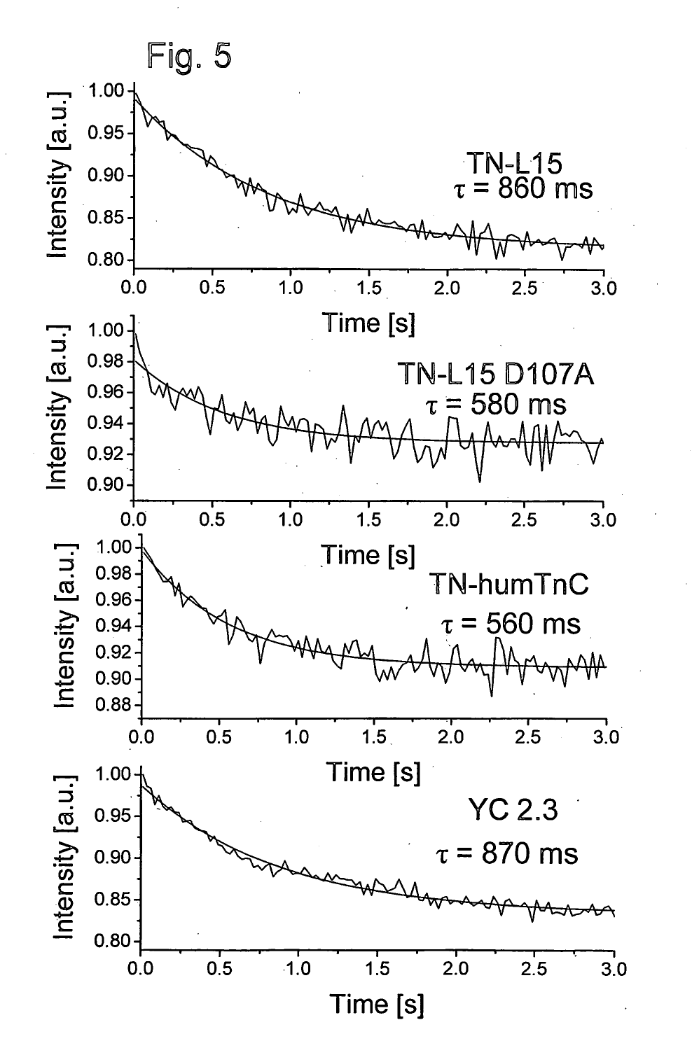

For measurements of dissociation kinetics, 6 μM purified protein in 10 mM MOPS pH 7, 200 mM KCl, 1 mM BAPTA, 1 mM free Mg²⁺ and 1 or 50 μM free Ca²⁺ (TN-L15 D107A: 50 μM or 300 μM free Ca²⁺) were mixed with 20 mM BAPTA (TN-L15 D107A: 35 mM BAPTA) in 10 mM MOPS pH 7,200 mM KCl and 1 mM free Mg²⁺; mixing dead time was 8 ms. In our experience on-rates of genetically encoded calcium probes never appeared to be a problem in experiments. However, slow dissociation rates are the main obstacle to follow fast changing signals. We therefore focused on measuring the dissociation rates of calcium bound to our indicator proteins. Samples were excited at 432 nm and emission monitored at 528 nm. Data sets from at least five experiments were averaged and rate constants derived from monoexponential curve fittings. Traces of individual dissociation experiments are shown in FIG. 5 As expected for first order reaction kinetics, these rates were independent of the chosen calcium concentration (data not shown). The r values obtained from the three selected constructs were 860 ms for TN-L15 (FIG. 5, top), 580 ms for TN-L15 D107A and 560 ms for TN-humTnC. In comparison with our proteins yellow cameleon 2.3 (YC2.3) displayed a calcium dissociation rate of 870 ms (FIG. 5, bottom).

### Example 3

### Functionality of TNC-Based Indicators in Live Cells

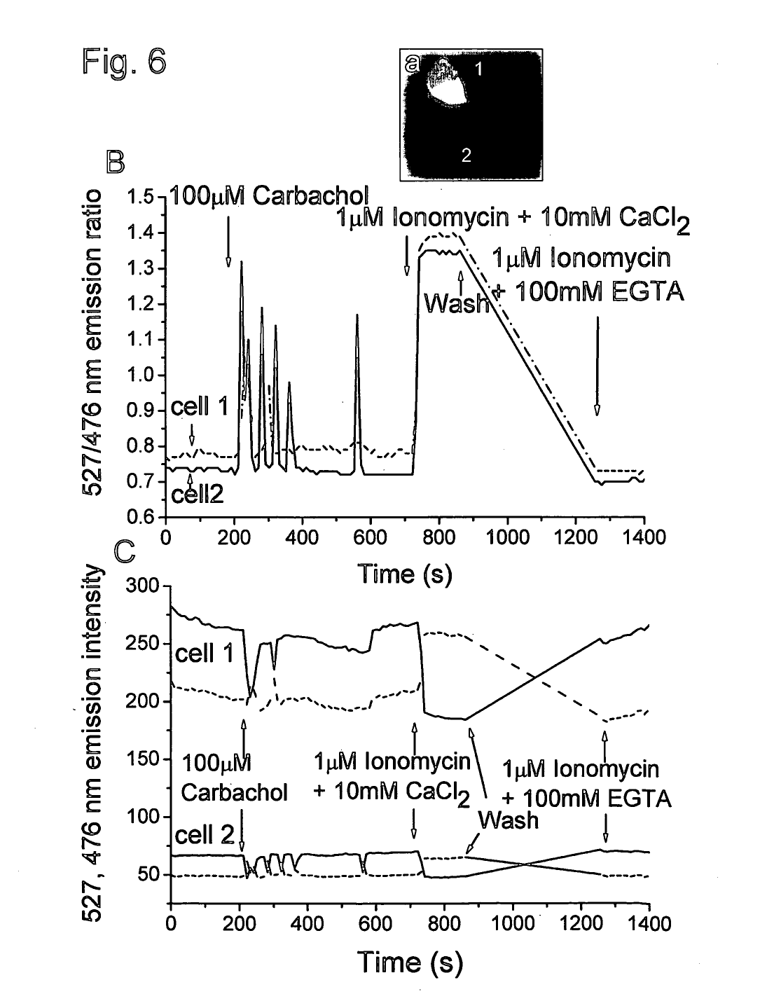

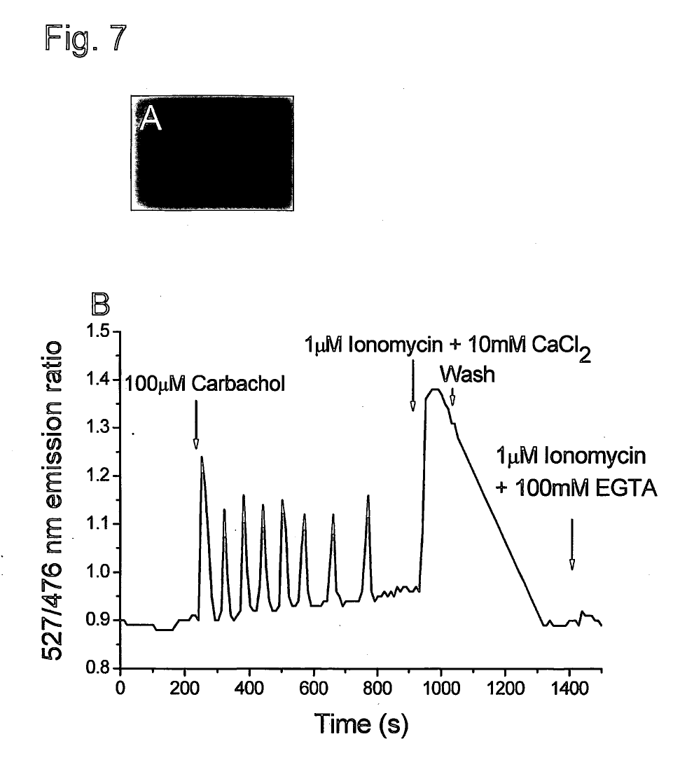

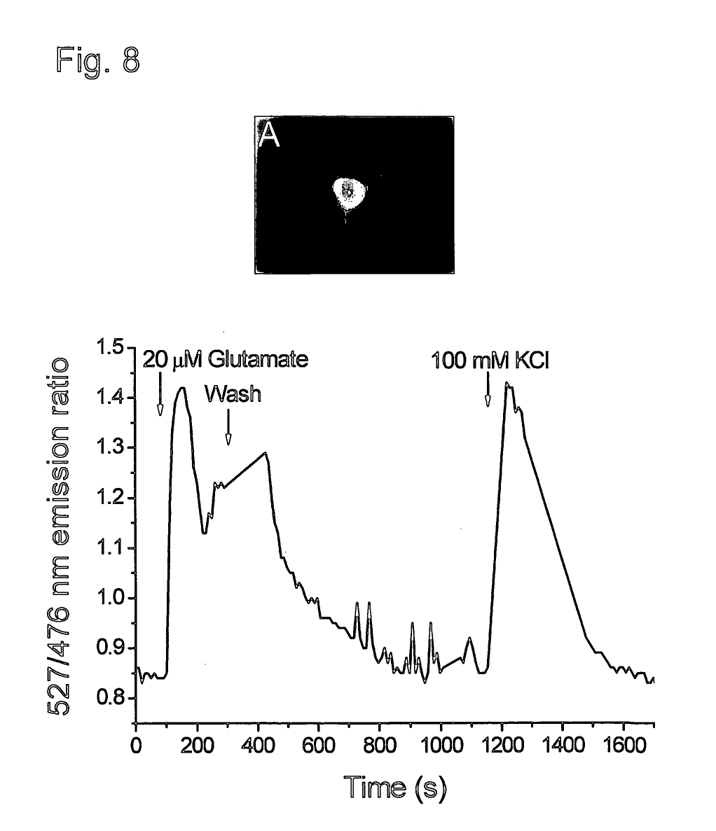

HEK-293 cells were transfected with lipofectin reagent (Invitrogen) and imaged two to four days later on a Zeiss Axiovert 35M microscope with a CCD camera (CoolSnap, Roper Scientific). Hippocampal neurons were prepared from 17 day old rat embryos, transfected by calcium phosphate precipitation 1 week after preparation, and imaged 2 days after transfection. The imaging setup was controlled by Metafluor 4.6 software (Universal Imaging). For ratio imaging, a 440/20 excitation filter, a 455 DCLP dichroic mirror and two emission filters (485/35 for CFP, 535/25 for Citrine) operated in a filter wheel (Sutter Instruments) were used. Constructs of the indicators with optimized Kozak consensus sequences for initiation of translation were expressed. Troponin C is a part of the troponin complex and usually not expressed as an isolated protein within the cytosol. It was therefore interesting and satisfying to see that our indicators showed good cytosolic expression. Fluorescence was distributed evenly and homogenously within the cytosol with no signs of aggregation (FIGS. 6A, 7A). The nucleus was excluded as expected for proteins with molecular weights of 69.7 and 72.5 kD, respectively for TN-L15 and TN-humTnC. In order to examine the function of the indicators inside cells we used the carbachol response of 293 cells that can be stimulated via muscarinic receptors. Responses of 293 cells expressing TN-L15 after stimulations with 100 μM carbachol can be seen in FIG. 6. Ratios (FIG. 6B) and intensity changes of the individual wavelengths (FIG. 6C) are depicted for two cells expressing different levels of the probe. In good agreement with the in vitro properties of the indicator, carbachol-induced oscillations of cellular free calcium were readily imaged, with repeated cycles of reciprocal intensity changes of CFP and Citrine. Imaging turned out to be dynamic and reproducible, and it was no problem to obtain Rmax and Rmin. TN-L15 was also functional in primary cultures of rat hippocampal neurons (FIG. 8). Responses to glutamate stimulation and depolarization with 100 mM KCl are seen in FIG. 8b. A response of HEK293 cells expressing TN-humTnC is shown in FIG. 7B. Maximal ratio changes within cells were 100% for TN-L15 and 70% for TN-humTnC, in accordance with the indicators' in vitro values. For comparison, the maximal ratio change obtainable with yellow cameleon 2.1 on our set-up was 70% (data not shown).

### Example 4

### Subcellular Targeting of TNC-Based Indicators and Functionality of Such Constructs

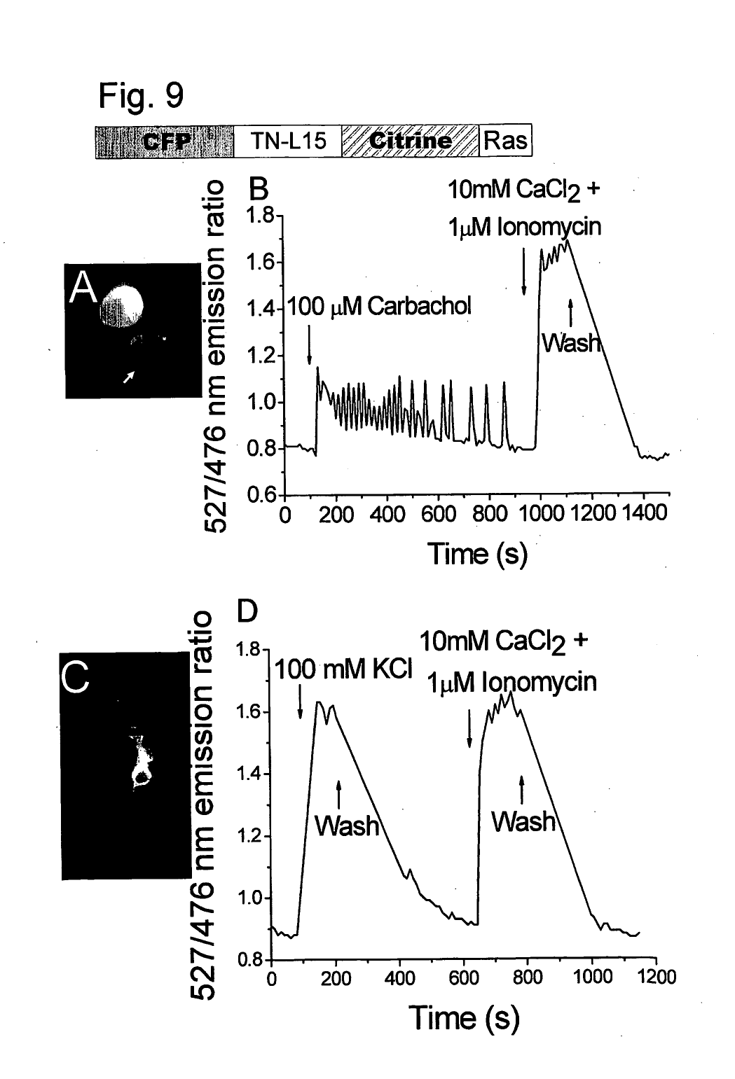

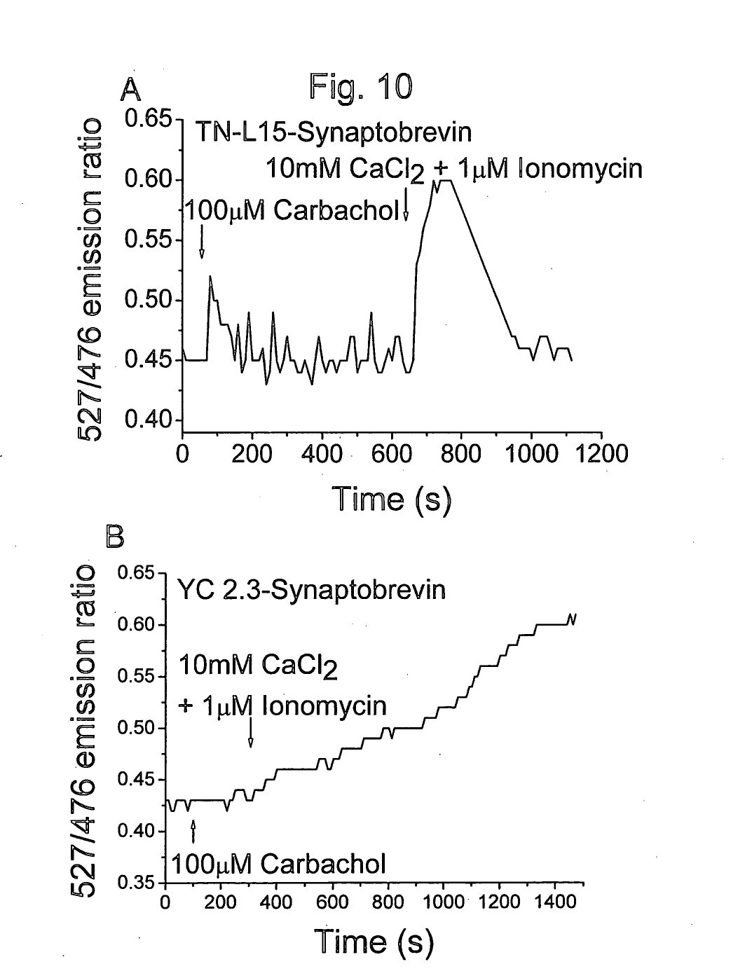

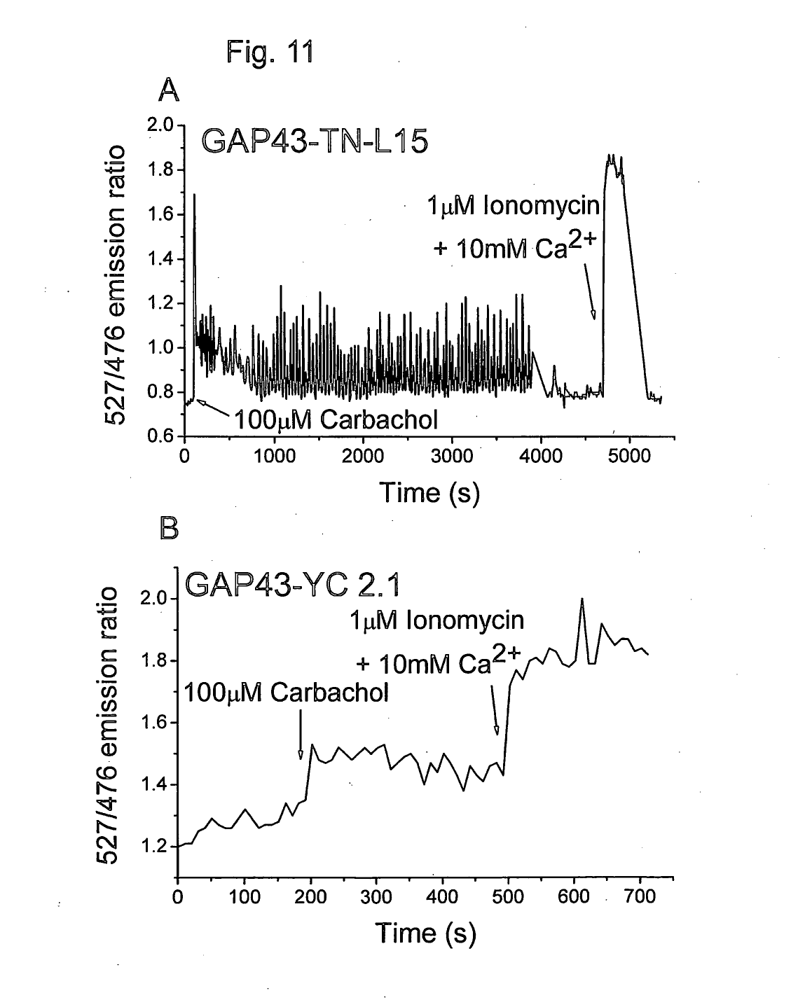

We next set out to evaluated the targeting properties of our new indicators within cells. In principle, one great potential of genetic probes is that they can be targeted to cellular organelles and microenvironments with the precision of molecular biology. Although most attractive, no functional labelings of membranes, pre- or postsynaptic structures or calcium channels have been reported previously. In our experience, these types of targetings were not functional when performed with calmodulin-based indicators (O. Griesbeck, unpublished observations and FIGS. 10, 11). We therefore used the membrane anchor sequence of c-Ha-Ras to target TN-L15 to the membrane (FIG. 9). Targeting was achieved by adding the membrane anchor sequence of c-Ha-Ras to the C-terminus of TN-L15. A scheme of the construct is depicted in FIG. 9. When expressed in 293 cells ring-shaped labeling of the plasma membrane was evident (FIG. 9A). For imaging we defined small regions following the contours of the membrane. Membrane-tagged TN-L15 readily reported agonist-induced increases in cytosolic calcium and had the same dynamic range as in cytosolic expression (FIG. 9B). When expressed in hippocampal neurons, TN-L15-Ras was saturated after stimulation with high potassium, probably due to the close vicinity to calcium channels in the plasma membrane (FIG. 9C, D). FIG. 11 shows a comparison of membrane-targeting of TN-L15 and Yellow Cameleon 2.1 (YC2.1) using the membrane targeting sequence of GAP43. The 20 N-terminal amino acid residues of GAP43 were added in the identical manner to the N-terminus of TN-L15 or YC2.1 in order to achieve targeting to the plasma membrane. The functionality of these constructs was tested in 293 cells. A. Imaging trace with GAP43-TN-L15. Long lasting calcium oscillations after stimulation with carbachol are visible. Finally calibration with ionomycin/10 mM CaCl2 and ionomycin/20 μM EGTA to obtain Rmax and Rmin verified that the indicator had its full dynamic range and full functionality when targeted to the plasma membrane. In contrast, GAP43-YC2.1 performed poorly under identical conditions as seen in FIG. 11B. No oscillations were detectable, and also calibration with ionomycin indicated a reduced dynamic range, suggesting that the indicator had lost significant features of its calcium binding properties on the pathway to membrane insertion. In another comparison of targeting properties we made use of fusion of TN-L15 and Yellow Cameleon 2.3 (YC2.3) to the presynaptic protein Synaptobrevin (FIG. 10). Fusions were done in the identical manner for both constructs. Fusion constructs were tested for functionality in 293 cells. A. Imaging trace of TN-L15-Synaptobrevin. Good responses to stimulation with carbachol and ionomycin were readily detectable. B. Imaging trace of YC2.3-Synaptobrevin. Within the fusion construct the indicator YC2.3 had largely lost its calcium sensitivity and binding properties. No responses to carbachol stimulation were seen. Ionomycin induced a sluggish rise of the ratio over several minutes that does not reflect the actual cytosolic rise in calcium levels after ionomycin treatment. The trace shown in B is an example chosen from 9 different imaging experiments, none of which elicited a response of this probe. Altogether these results clearly demonstrate the superiority of troponin-based indicators, especially under the experimental conditions of membrane targeting.

### Example 5

### A Transgenic Mouse Line Expressing TN-L15 in the Cytosol of Neurons

XhoI restriction sites were added to both sides of the TN-L15 sequence by PCR amplification using a suitable primer pair, and the indicator was then cloned into the Xhol-site of the mouse Thy-1.2 expression cassette contained in a pUC18 vector (Caroni P., J Neuroscience Methods 71 (1997) 3-9). The transgene insert was then stripped of all vector sequences by restriction with EcoRI/PvuI and purified via agarose gel electrophoresis and electroelution of the DNA fragment into a dialysis bag (after Sambrook and Russell, “Molecular Cloning” 3rd ed. (2001), CSHL Press, Cold Spring Harbor, chapter 5). In order to further purify the DNA of all contaminants, an ion exchange chromatography was performed using small disposable Elutip-D Minicolumns (Schleicher & Schüll). The column was equilibrated in a low salt buffer (0.2 M NaCl, 20 mM Tris HCl, 1.0 mM EDTA, pH 7.4), the DNA obtained from the electrolution procedure applied to the column, washed with low salt buffer and then eluted with a high salt buffer (1.0 M NaCl, 20 mM Tris HCl, 1.0 mM EDTA, pH 7.4) The purified DNA fragment was then used for the creation of transgenic animals by the DNA microinjection method into pronuclei of FVB mouse oocytes (Taketo M. “FVB/N: an inbred mouse strain preferable for transgenic analyses”, Proc Natl Acad Sci USA (1991)₈₈(6):2065-9; Manipulating the Mouse Embryo: A Laboratory Manual 3rd ed.; Nagy et al. eds. (2002), CSHL Press, Cold Spring Harbor). Founder animals were screened for CFP/YFP by PCR of genomic DNA obtained from tail lysates using the Proteinase K/isopropanol precipitate method (“Molecular Cloning” 3rd edition, Sambrook and Russell (2001), CSHL Press, Cold Spring Harbor, chapter 6), and PCR-positive founders were crossed with wildtype C57BL/6 mice.

Fluorescent protein expression was visualized in fixed brain slices by immersing brains of PCR-positive animals in 4% paraformaldehyde/PBS for 2 h and 30% Sucrose/PBS overnight. The tissue was then frozen in Tissue-Tek mounting medium (Sakura) and cut into slices of 50 μm thickness on a Microm HM400 freezing microtome. The distribution of calcium indicator protein was determined by immunostaining with polyclonal anti-GFP rabbit antibodies (RDI) and a TRITC-labelled secondary swine antibody (DakoCytomation). Immunostained slices were mounted on glass slides and analysed with an upright fluorescence microscope. Fluorescence of indicator protein in acute brain slices was observed by cutting brains of positive animals into 350 μm thick slices using a vibratome and subsequent fluorescence microscopy of living slices immersed in oxygenated artificial cerebrospinal fluid (118 mM NaCl, 3 mM KCl, 1 mM MgCl₂, 1 mM CaCl₂, 25 mM NaHCO₃, 1 mM NaH₂PO₄, 30 mM Glucose; pH 7.4).

Organotypic slices for fluorescence imaging were prepared after the protocol published by Stoppini et al., J Neuroscience Methods, 37 (1991), 173-182: hippocampi from 4 day old mice were cut into 400 μm thick slices with a vibratome, washed, and placed on culture plate filters (Millipore). Those filters were then cultured for 2 weeks in 6-well plates containing medium (50% BME, 25% horse serum, 25% HBSS with 1 mM Glutamin and 5 mg/mg Glucose; GIBCO). Imaging of the slices was done on a fluorescence setup as described in EXAMPLE 3; during imaging, slices were kept in HBSS and held in place at the bottom of the dish with the help of a platinum ring. Calcium responses were evoked by depolarizing the neurons with potassium; for this purpose, the KCl concentration of the HBSS solution was raised to 50 mM while images were taken at an interval of 5 s.

1-26. (canceled)

27. A modified Ca2+-binding polypeptide comprising:
a) a first chromophore of a donor-acceptor-pair for FRET (Fluorescence Resonance Energy Transfer);
b) a Ca2+-binding polypeptide with an identity of at least 80% to a 30 amino acid long polypeptide sequence of human troponin C or chicken skeletal muscle troponin C or drosophila troponin C isoform 1; and
c) a second chromophore of a donor-acceptor-pair for FRET.

28. The polypeptide of claim 27, wherein the first chromophore is a fluorescent polypeptide capable of serving as a donor-chromophore in a donor-acceptor-pair for FRET and the second chromophore is a fluorescent polypeptide capable of serving as an acceptor-chromophore in a donor-acceptor-pair for FRET.

29. The polypeptide of claim 28, wherein the modified polypeptide is a fusion polypeptide wherein the order of the three linked polypeptides starting from the N-terminus of the fusion polypeptide is a)-b)-c) or c)-b)-a).

30. The polypeptide of claim 27, wherein the first chromophore is selected from the group consisting of CFP, EGFP, YFP, DsFP 483, AmCyan, Azami-Green, Cop-Green and As499, particularly wherein the first chromophore is CFP.

31. The polypeptide of claim 27, wherein the second chromophore is selected from the group consisting of YFP, DsRed, zFP 538, HcRed, EqFP 611, Phi-Yellow and AsFP 595.

32. The polypeptide of claim 31, wherein the second chromophore is YFP.

33. The polypeptide of claim 27, wherein the Ca2+-binding polypeptide comprises at least one Ca2+-binding EF-hand.

34. The polypeptide of claim 27, wherein the Ca2+-binding polypeptide comprises a polypeptide sequence having at least 60% identity to: (1) amino acids 15 to 163 of chicken skeletal muscle troponin C or (2) amino acids 1 to 161 of human cardiac troponin C or (3) amino acids 5 to 154 of drosophila troponin C isoform 1.

35. The polypeptide of claim 27, further comprising glycine-rich linker peptides N-terminal or C-terminal to polypeptide b).

36. The polypeptide of claim 27, further comprising a localization signal.

37. The polypeptide of claim 36, wherein the localization signal is a nuclear localization sequence, a nuclear export sequence, an endoplasmic reticulum localization sequence, a peroxisome localization sequence, a mitochondrial import sequence, or a mitochondrial localization sequence, a cell membrane targeting sequence.

38. The polypeptide of claim 37, wherein the localization signal is a cell membrane targeting sequence mediating localization to pre- or postsynaptic structures.

39. The polypeptide of claim 27, which exhibits a ratio change upon Ca²⁺— addition of more than 30%, preferably from 50% to 200%, more preferably from 80% to 180%, and most preferably from 100% to 150%.

40. The polypeptide of claim 27, which has a Kd for Ca²⁺ of from 50 nM to 400 μM, preferably of from 100 nM to 100 μM, and most preferably of from 250 nM to 35 μM.

41. The polypeptide of claim 29, selected from the group consisting of the polypeptides of SEQ ID NO: 2, 4, 6, 8, 10, 12, 14, 16, 18, 32, 34, and 42, preferably 2, 4, 34, or 42.

42. A nucleic acid molecule comprising a nucleic acid sequence encoding a polypeptide according to claim 29, preferably a nucleic acid sequence of SEQ ID NO: 1, 3, 33, or 41.

43. An expression vector containing the nucleic acid molecule of claim 42, preferably further comprising expression control sequences operatively linked to a nucleic acid encoding a polypeptide, wherein the polypeptide is a modified Ca²⁺-binding polypeptide comprising:
a) a first chromophore of a donor-acceptor-pair for FRET (Fluorescence Resonance Energy Transfer), wherein the first chromophore is a fluorescent polypeptide capable of serving as a donor-chromophore in a donor-acceptor-pair for FRET;
b) a Ca²⁺-binding polypeptide with an identity of at least 80% to a 30 amino acid long polypeptide sequence of human troponin C or chicken skeletal muscle troponin C or drosophila troponin C isoform 1;
c) a second chromophore of a donor-acceptor-pair for FRET, wherein the second chromophore is a fluorescent polypeptide capable of serving as an acceptor-chromophore in a donor-acceptor-pair for FRET; and
d) wherein the modified polypeptide is a fusion polypeptide wherein the order of the three linked polypeptides starting from the N-terminus of the fusion polypeptide is a)-b)-c) or c)-b)-a).

44. A host cell, particularly a mammalian, non-human cell, inside or outside of the animal body or a human cell outside of the human body, comprising a polypeptide according to claim 29.

45. A host cell, particularly a mammalian, non-human cell, inside or outside of the animal body or a human cell outside of the human body, comprising a nucleic acid according to claim 42.

46. A host cell, particularly a mammalian, non-human cell, inside or outside of the animal body or a human cell outside of the human body, comprising an expression vector according to claim 43.

47. A transgenic animal comprising a polypeptide according to claim 29.

48. A transgenic animal comprising a nucleic acid according to claim 42.

49. A transgenic animal comprising an expression vector according to claim 43.

50. A transgenic animal comprising a host cell according to claim 44.

51. A method for the detection of changes in the local Ca²⁺-concentration comprising the following steps:
a) providing a cell or a subcellular membranous fraction of a cell comprising a Ca2+-binding polypeptide according to claim 27;
b) inducing a change in the local Ca2+-concentration; and
c) measuring FRET between the donor and the acceptor chromophore of the donor-acceptor-pair of said polypeptide according to claim 27, which is indicative of the change in the local Ca²⁺-concentration.

52. The method of claim 51, wherein the cell of step a) is a host cell, particularly a mammalian, non-human cell, inside or outside of the animal body or a human cell outside of the human body, comprising a polypeptide according to claim 29.

53. The method of claim 51, wherein the subcellular membranous fraction is an organelle, in particular a mitochondrium, a peroxisome or a nucleus, or a membrane fraction derived from a membrane-bound organelle, in particular derived from the cell membrane.

54. The method of claim 51, wherein the Ca²⁺-binding polypeptide is targeted to the inner surface of the cell membrane.

55. The method of claim 51, wherein step b) is effected by administering an ex-tracellular stimulus, in particular by adding a small chemical compound or a polypeptide to the extracellular side of the host cell.

56. A method for the detection of the binding of a small chemical compound or a polypeptide to a Ca2+-binding polypeptide with an identity of at least 80% to a 30 amino acid long polypeptide sequence of human troponin C or chicken skeletal muscle troponin or drosophila troponin C isoform 1, comprising the following steps:
a) providing a Ca²⁺-binding polypeptide according to claim 27;
b) adding a small chemical compound to be tested for binding or a polypeptide to be tested for binding; and
c) determining the degree of binding by measuring FRET between the donor and the acceptor chromophore of the donor-acceptor-pair of said polypeptide according to claim 27.

57. The method of claim 56, wherein the Ca²⁺-binding polypeptide is derived from human troponin C, and particularly is SEQ ID NO: 4.

58. A method of using a polypeptide according to claim 27, comprising the step of detecting changes in the local Ca²⁺-concentration close to a cellular membrane.

59. The method of claim 57, wherein the polypeptide comprises a localization sequence, and particularly comprises a cell membrane targeting sequence, most preferably a cell membrane targeting sequence mediating localization to the cell membrane of pre- or postsynaptic structures.

60. A diagnostic composition suitable for the detection of changes in the local Ca2+-concentration close to a cellular membrane, said composition comprising a polypeptide according to claim 27.
